SEQUENCE LISTING (41 sequences)

SEQ ID NO: 1 - protein, 260 aa, Mus musculus
VNKVSNVFEDVEFCVMSGLDGYPKADLENRIAEFGGYIVQNPGPDTYCVIAGSENVRVKNIISSDKNDVVKPEWLLECFKTKTCVPWQPRFMIHMCPSTKQHFAREYDCYGDSYFVDTDLDQLKEVFLGIKPSEQQTPEEMAPVIADLECRYSWDHSPLSMFRHYTIYLDLYAVINDLSSRIEATRLGITALELRFHGAKVVSCLSEGVSHVIIGEDQRRVTDFKIFRRMLKKKFKILQESWVSDSVDKGELQEENQYLL

SEQ ID NO: 2 - protein, 56 aa, Mus musculus
DCYGDSYFVDTDLDQLKEVFLGIKPSEQQTPEEMAPVIADLECRYSWDHSPLSMFR

SEQ ID NO: 3 - protein, 547 aa, Mus musculus
IQCIQSVYTSKIISSDRDLLAVVFYGTEKDKNSVNFKNIYVLQDLDNPGAKRVLELDQFKGQQGKKHFRDTVGHGSDYSLSEVLWVCANLFSDVQLKMSHKRIMLFTNEDDPHGRDSAKASRARTKASDLRDTGIFLDLMHLKKPGGFDVSVFYRDIITTAEDEDLGVHFEESSKLEDLLRKVRAKETKKRVLSRLKFKLGEDVVLMVGIYNLVQKANKPFPVRLYRETNEPVKTKTRTFNVNTGSLLLPSDTKRSLTYGTRQIVLEKEETEELKRFDEPGLILMGFKPTVMLKKQHYLRPSLFVYPEESLVSGSSTLFSALLTKCVEKKVIAVCRYTPRKNVSPYFVALVPQEEELDDQNIQVTPGGFQLVFLPYADDKRKVPFTEKVTANQEQIDKMKAIVQKLRFTYRSDSFENPVLQQHFRNLEALALDMMESEQVVDLTLPKVEAIKKRLGSLADEFKELVYPPGYNPEGKVAKRKQDDEGSTSKKPKVELSEEELKAHFRKGTLGKLTVPTLKDICKAHGLKSGPKKQELLDALIRHLEKN

SEQ ID NO: 4 - protein, 306 aa, Mus musculus
MEDLRQYMFSSLKNNKKCTPTEAQLSAIDDLIDSMSLVKKNEEEDIVEDLFPTSKIPNPEFQRLYQCLLHRALHLQERLPPIQQHILNMLDPPTEMKAKCESPLSKVKTLFPLTEVIKKKNQVTAQDVFQDNHEEGPAAKKYKTEKEEDHISISSLAEGNITKVGSVNPVENFRFLVRQKIASFEEASLQLISHIEQFLDTNETLYFMKSMDCIKAFREEAIQFSEEQRFNSFLEALREKVEIKQLNHFWEIVVQDGVTLITKDEGPGSSITAEEATKFLAPKDKAKEDTTGPEEAGDVDDLLDMI

SEQ ID NO: 5 - protein, 199 aa, Clostridium spec. 7_2_43 FAA
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 6 - DNA, 597 bp, Clostridium spec. 7_2_43 FAA
gaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatac

SEQ ID NO: 7 - protein, 587 aa, Clostridium spec. 7_2_43 FAA
MINIIDVNNKTIRTFGWVQNPSNFESLKKVVAIFDNTSKTYNELKDKKIKKLVDERDGQKELLNALNANPLKIKYCNLVGTSFTPRSSARCNGIVQATVKGQRKEFIDDWSSDNFVRWAHALGFIKYNYDTDTFEITDVGRKYVQSEDDSNEESTILEEAMLSYPPVARVLTLLSNGEHLTKYEIGKKLGFVGEAGFTSLPLNVLIMTLATTDEPKEKNKIKTDWDGSSDKYARMISGWLVKLGLLVQRPKLVTVDFGGELYSETIGHAYMITDRGLKAVRRLLGINKVARVSKNVFWEMLATKGIDKNYIRTRRAYILKILIESNKVLTLEDIKGKLKLASINESINTIKDDINGLINTGINIKSETTGYKIYDSINDFIIPKTGDTEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 8 - DNA, 6606 bp, Artificial sequence
  Vector Rab38-cht
caccgcattaccctgggcgttgaaaccgaagaagacctggatttgaaataggcgttttctttacatttctaaagtgggactcctcacttgtaaaaggaaaaataatgatacttttaagacttccaggatgactaaatggtgtgtatgagaagatttataaacatctgccgctacttacaatgataagaccacttgtgtgttgttcagcttggagaatttaggataggagtggaggctgaaagaaaagtaagcccttagcatttcctctcaggtggcctctactttaggtcattaacagttgaataggcgctaagagatagcattaccactttatagaagcccaggcaaaaggagattaaagggtttgcctaaattctttcaactctaagggccagagaagacctaagtctactgctttgctgtttctcaaggtctccccaactttacaacactgtgtgggtggcaacagggcttaatagcctcagaagacctgggtatttttcgacactcagttctctccccggcagaacgtggaaaacaaaatccacataagtttgtgtcatggacgggaggcgagagaaaaatctctgtgaaaggagtaaagcactgtgcaaataccagcttgacaggcagtagcactggggtcccgggtcctttagcttccagtcccaggagttgctcttgtctcctcccactctggagtccgcagagtaggaaggaggattaaacccgggggaggagttccgcaccagctccctatcctgcgccagcacgcctagcctaagcgcccacatagagctccggtctccgtcggtgcccagccccggctgtgcttcccagagcaagctccaggctccgcaagacccgcgggcctccaggatgcagacacctcacaaggagcacctgtacaagctgctggtgatcggcgacctggtagtgggcaagaccagcattatcaagcgctatgtgcatcaaaatttctcctcgcactatcgagccaccattggtgtggacttcgcgctgaaggtgctccactgggacccagagacggtggtgcgcttgcagctctgggacattgctggtgagcgatcagagcagcgcgcaacgggtgagggtggagtgagccagtgaggagttcgggggtgaaggttcggggagtggaaaatgacttttcagtcggttccagtcccgggacccttgagtgcaatcaagcaggagatccggatcgcctgggcgctccactcttggaaagtttggcttaatggcttggaaacctgatttcaaagaaatggaagtgttttcttttctttctttccttttttttttttttttttttcttttgctgttgtttctgttggagtcgtccccactctacctgtaacttctagataacttcgctggctctcactggctgtgagaaagcgaaccactttctcctgggattcttgggtgcagagaaggctgtcgcctggactcacaaggagattgtagtcgcattcttgtttcattctagtccttttctggacacaggtagccgcgacttggcccagagtatctcacgtggctttcatccttcgtgtttagaggggaagcccctaggaaatttaagaaggagcaggattatcttaggaatttagtttctttcaaatctcactactatcatctccttgcttattggcctcttcagtcagaaaaatttgagatgctaaatttgtatacatctagaacgaactatctcttctcactccactcccctcttccccatctctcttccgtctccctccatccttggctatctcttcttcactttccatttcaaacaggagactgtgtatgttttttaggaaaacattaaaaaaaaaaccacaaaaacaaaaacaaaacggagacagggtcccgtcatgtaactctgctaacctatatcaagctgaccttgacctcatagagacccacttgcctctgcctccctagaggcaagggtcggggttatggtgatgttaatgtcgtttgctttaagattccttgatttgatcttggtgtattttttgagaaatctaaagtatgaaatcagagtttgactaacagcttctaccagctcctagccacaataaagactgaggcaggctatagttagtgctcaatactgggtcctacctggctgcttgtaacctgggcatgcctagcattctagatgctaactcaccaaagcagtagcattttaagctgcaaatggctaggcagcgacagctcaagaatcttcttgctttggagttttaaactccaatgagattttccatgatccctttcaaataaccctacttaatctctcttcatagcccacagtaccaagaagcctttgataagctctggattgaaaagaagcagttctttttcaaaagatgtgctcatttgaactagtgcatttccctggaaacactttgccaggacttgagatgggcactaagaaggaaaattcctcaaaggacatgtacagtcttgagatgcattcgcttctgtagccatgagcttgctggtcttgagataaggttagttggtgtagctaggttcatggtttggagtctttggcagttctagagaagcatgagctattagagacttggagattgcatcaagtagagccttttgagcttttcactgtgtacctgggccctctgtcgctgcacgttttagtgtctgaaatgtctttcagctgtagcagttttctcgggaccccagtttaaaatagcttactgtttaaaagatgtagctgtagctagcattattgaactagcataattatagtctaaatagcattatgtcttcagccttgttatatgttggtgagttttagtttcctcttctaaacgggaagaacagaaagatgtaatgattctgagcttccagagtgagacacctctagagagaaataccttcttctgaagactaccgtgtgattacagataaattctgatatctttgtttagcttttgatatctataaacagggagtgtattttatctctccaaatgagagaagaataaacaataatgcaaggtaaaggcaatagtgctacactctaggagttaccactctttgtacatttatttataaatactaagcaagaggaacatgccatacatacactgactaagtcctaacaagtggcagttcttatatcacacatttatcttgccctcaaatgccagtccagcatcagtttagtctcatgcatttggcagcataaggcagtttgagttccacacttgctctcagaagcaatttaactcccacacttgggaatcctttcctaagccacagtttcagaccaaagttttggtgaaggctataatcacagaagtctgcacaagtagggagtctgaaggatctgagctccattcagcagtcagagcggcatccaaccccaaggtaatgctcagctcactttgataacttcaagctcaaaggccctgaactgctgagttggaggttgaaagatgtttgggtaaaagcaaggtaattggcggataggatggttgtaacgtaattgtttcaagttgtattagagacctctgggttctaaggggatatgaaatccaacctccactctccactgagattcaagttaggttaagtatgcctttgagtaccctcaagtcacagcatgccactctccttttcttaactctaatatgtatctataaagaacgggtagtagtcaactgagtcgacggtatcgataagcttgatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagct

SEQ ID NO: 9 - DNA, 202 bp, Mus musculus
atgcagacacctcacaaggagcacctgtacaagctgctggtgatcggcgacctgggtgtgggcaagaccagcattatcaagcgctatgtgcaccaaaacttctcctcgcactaccgggccaccattggtgtggacttcgcgctgaaggtgctccactgggacccagagacggtggtgcgcttgcagctctgggacattgctg

SEQ ID NO: 10 - DNA, 39 bp, Artificial sequence
  Primer RabCht-1
ctcaccgcattaccctgggcgttgaaaccgaagaagacc

SEQ ID NO: 11 - DNA, 37 bp, Artificial sequence
  Primer RabCht-2
gataatgctggtcttgcccactaccaggtcgccgatc

SEQ ID NO: 12 - DNA, 82 bp, Artificial sequence
  Primer RabCht-3
gtgggcaagaccagcattatcaagcgctatgtgcatcaaaatttctcctcgcactatcgagccaccattggtgtggacttcg

SEQ ID NO: 13 - DNA, 40 bp, Artificial sequence
  Primer RabCht-4
gcttatcgataccgtcgactcagttgactactacccgttc

SEQ ID NO: 14 - DNA, 21 bp, Artificial sequence
  Primer cht-Ex1F
ggcctccaggatgcagacacc

SEQ ID NO: 15 - DNA, 21 bp, Artificial sequence
  Primer cht Ex1R
ccagcaatgtcccagagctgc

SEQ ID NO: 16 - DNA, 35 bp, Artificial sequence
  Primer lig4-1
gactacgcgtgtaaacaaagtttccaatgtatttg

SEQ ID NO: 17 - DNA, 40 bp, Artificial sequence
  Primer lig4-2
acgtacgcgtgcggccgcctaaagcaaatactggttttcc

SEQ ID NO: 18 - DNA, 6569 bp, Artificial sequence
  pCAG-venus-lig4-bpA
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgcccaagaagaagaggaaggtgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggcggaggcggaggcggaggcacgcgtgtaaacaaagtttccaatgtatttgaagatgttgagttttgtgttatgagtggattagatggttatccaaaggctgacctagagaacagaattgcagaattcggtggttatatagtacagaatccaggcccggatacatactgtgttattgcaggttctgagaacgttagagtgaaaaacattatttcttcagataaaaatgatgttgtcaagcccgagtggcttttagagtgttttaagacaaaaacatgcgtgccgtggcaacctcgctttatgattcacatgtgcccgtcgacaaagcagcattttgcccgtgagtatgactgctatggtgatagctattttgttgacacagatttggatcaattgaaagaagtgtttctaggaattaaacccagtgagcagcagactcctgaagaaatggcccctgtgattgctgacttagaatgtcgttattcctgggaccactctcctctcagtatgtttcgacattacaccatttatttggacttgtatgctgttattaatgacttgagttccagaattgaagccacgagattaggtattacagcccttgagctgcggtttcatggagcaaaggtggtttcctgcttatctgaaggggtatctcatgttatcattggggaggatcagagacgagttactgactttaaaatattcagaagaatgcttaagaaaaagtttaaaatcctgcaagaaagttgggtgtccgattcagtagacaagggcgaactgcaggaggaaaaccagtatttgctttaggcggccgcacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 19 - protein, 56 aa, Mus musculus; identical to SEQ ID NO: 2

SEQ ID NO: 20 - DNA, 5906 bp, Artificial sequence
  plasmid pCAG-E4ORF6-bpA
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagtaggcggccgcacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 21 - DNA, 6512 bp, Artificial sequence
  plasmid pCAG-E1b55K-bpA
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgataggcggccgcacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 22 - DNA, 66 bp, Artificial sequence
  >primerE4-1
gatcttaattaataatacgactcactataggggccgccaccatgactacgtccggcgttccatttg

SEQ ID NO: 23 - DNA, 46 bp, Artificial sequence
  >primerE4-2
gatcacgcgtgcggccgcctactacatgggggtagagtcataatcg

SEQ ID NO: 24 - DNA, 63 bp, Artificial sequence
  >primerE1b-1
gatcttaattaataatacgactcactataggggccgccaccatggagcgaagaaacccatctg

SEQ ID NO: 25 - DNA, 46 bp, Artificial sequence
  >primerE1b-2
gatcacgcgtgcggccgcctatcaatctgtatcttcatcgctagag

SEQ ID NO: 26 - DNA, 6684 bp, Artificial sequence
  pCAG-Ku70(62-609)-bpA
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgcccaagaagaagaggaaggtgatccagtgtatccagagtgtgtacaccagtaagatcataagcagcgatcgggatctcctggcagtggtgttctatggaaccgagaaagataaaaattcagtgaatttcaaaaatatttatgtcttacaagatttggacaacccaggcgctaagcgagtgctagagctcgaccagtttaagggacaacaggggaagaagcacttccgagacacggttggccatgggtctgactactctttgagtgaagtgctctgggtctgtgccaacctcttcagcgacgtccagctcaagatgagtcacaagaggatcatgctgttcaccaatgaagacgacccccatggccgtgacagtgctaaagccagccgggccaggaccaaagccagcgacctccgggacactgggatcttccttgacttgatgcatctgaagaagccaggaggctttgatgtatccgtgttctacagggacatcatcaccaccgctgaggacgaggaccttggggttcacttcgaggagtcaagcaagctggaagacctgctaaggaaggttcgagccaaggagaccaaaaagcgagttctgtccaggttaaagtttaagctcggtgaagacgtagtactcatggtgggcatttataacttggtccagaaagctaacaagccttttccagtgagactctatcgggaaacaaatgaaccagtgaaaaccaagacaaggacttttaatgtaaacaccggcagtctactcctgcctagtgacaccaagcggtctctgacttacgggacacgtcagattgtgctggagaaagaggagacagaggagctgaagcggtttgatgagccaggtttgatcctcatgggctttaagcccacggtgatgctgaagaagcagcactacctgaggccctctctgttcgtgtacccagaggagtccctggtcagtgggagctcaaccttgttcagcgctctgctcaccaagtgtgtggagaagaaggtcatagcagtgtgtagatacacaccccggaagaacgtctccccgtattttgtggctttggtgccccaggaagaggagctggatgatcagaacattcaggtgactccaggaggcttccagcttgtcttcctcccttatgccgatgacaagcggaaggtgccctttactgagaaggtgacggccaaccaggagcagatagacaagatgaaggccattgttcaaaagctccgcttcacatacaggagcgacagttttgagaatccagtcctgcagcagcacttccgcaacctggaggccctagctttggacatgatggagtcggagcaagtggtagatctgacactacccaaggttgaagccataaagaaaagactgggttccctggcagatgagtttaaagaacttgtctatcctccaggttataatcccgagggaaaagttgccaagagaaaacaagatgatgaaggttctacgagtaaaaagcccaaggtagagttatcagaagaagagctgaaggcccattttcgtaagggcacactgggtaagctcactgtacctacactgaaggacatatgcaaggctcatgggcttaagagtgggccgaagaagcaggaactgctagatgctcttatcagacacttggagaagaactgaggatccacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 27 - DNA, 5961 bp, Artificial sequence
  pCAG-Ku80(427-732)-bpA
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgcccaagaagaagaggaaggtgatggaagacttgcggcaatacatgttttcctcgctgaaaaacaataagaagtgcactcccacagaggcccagctgagcgctattgatgatctgattgattccatgagcttggtaaagaaaaacgaggaagaagacattgttgaagatttgtttccaacctccaaaattccaaatcctgaatttcagcgattgtaccagtgtctgctgcatagagccttacatctccaggagcggctgcccccgattcagcagcacattttgaatatgctggatccccccactgagatgaaagcaaaatgtgagagtccactctctaaagtaaagacccttttccctctcacagaagtcatcaagaaaaagaaccaagtgactgctcaggacgttttccaagacaatcatgaagaggggcccgctgctaaaaaatataaaactgagaaagaagaagatcacatcagcatctccagcctggcagaagggaacatcaccaaggttggaagtgtgaatcctgttgaaaacttccgtttcctagtaagacagaagattgccagctttgaggaagcgagtctccagttaataagtcacatcgaacaatttttggataccaatgaaacactgtattttatgaaaagtatggactgcatcaaagctttccgggaggaggccattcagttttcagaagaacagcgcttcaacagcttcctggaagcccttcgagagaaagtggaaattaagcaattaaatcatttctgggaaattgttgttcaggatggagttactctgatcaccaaagacgaaggcccaggaagctctatcacagctgaggaagccacaaagtttctggcccccaaagacaaagcaaaagaagatacaacgggacctgaagaagctggtgatgtggatgatttactggacatgatatagctgcagacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 28 - DNA, 89 bp, Artificial sequence
  primerKu70-1
gatcttaattaataatacgactcactataggggccgccaccatgcccaagaagaagaggaaggtgatccagtgtatccagagtgtgtac

SEQ ID NO: 29 - DNA, 38 bp, Artificial sequence
  primerKu70-2
gatcacgcgtggatcctcagttcttctccaagtgtctg

SEQ ID NO: 30 - DNA, 89 bp, Artificial sequence
  primerKu80-1
gatcttaattaataatacgactcactataggggccgccaccatgcccaagaagaagaggaaggtgatggaagacttgcggcaatacatg

SEQ ID NO: 31 - DNA, 40 bp, Artificial sequence
  primerKu80-2
gatcacgcgtctgcagctatatcatgtccagtaaatcatc

SEQ ID NO: 32 - DNA, 6453 bp, Artificial sequence
  pCAG-Tal-IX-Fok
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttatgagacgacgtctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 33 - DNA, 7858 bp, Artificial sequence
  pCAG-RabChtTal-1
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaagttcgttccaccgtcgctcagcaccatgaagcactggtggggcacggtttcacacacgcccatattgtggctctgtctcaacatcccgctgcactcgggactgtggccgtcaaatatcaggacatgatcgccgctctgcctgaggcaacccacgaagccattgttggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttacacccgaacaggtggtggccatcgctagtaacattgggggcaaacaggctctggaaacagtacagcggctgttacctgtgctgtgccaggctcatggcctcacacctcagcaggtcgtcgcaatcgcctccaatggcggagggaagcaggccctggaaacggtgcagagactgttaccagtgctgtgccaggcccatggcctaacaccccagcaggtggtggccatcgccagccacgacggcggcaagcaggccctggaaaccgtgcagaggctgctgcctgtgctgtgccaggctcatggcctgacacctgagcaggtcgtcgccatcgccagcaacatcggcggcaagcaggccctggaaaccgtgcagaggctgctgccagtgctgtgccaggcccatggcttaacacccgaacaggtggtggccatcgcttctaatattgggggcaagcaggccctggaaacagtccagagactgttgcctgtgctgtgccaggctcatggcttgacacctcagcaggtcgtcgctatcgcctctaataatgggggcaagcaggctctggagacagtacagcgcctgttaccagtgctgtgccaggcccacgggctcacaccccagcaggtggtggcaatcgcttcccatgacggagggaaacaggctctggaaacggtccagaggctgctccctgtgctgtgccaggctcacggtctaacaccccagcaggtggtggccattgctagcaacaatgggggcaagcaggctctggagacagtgcagcgcctgctgcctgtgctgtgccaggctcatggcctcacacctcagcaggtcgtcgccatcgccagccacgacggcggcaagcaggccctggaaaccgtgcagaggctgctgccagtgctgtgccaggcccatggcctaacaccccagcaggtggtggcaatcgcctccaatggcggagggaagcaggccctggaaacggtgcagagactgttacctgtgctgtgccaggctcatggcctgacacctgagcaggtcgtcgctatcgctagcaatatcggagggaagcaggctctggaaactgtccagcgcctgctcccagtgctgtgccaggcccatggcttaacaccccagcaggtggtggcaattgctagcaatggcggagggaagcaggccctggagactgtccagagactgctacctgtgctgtgccaggctcatggcttgacacctcagcaggtcgtcgctatcgcctctaataatgggggcaagcaggctctggagacagtacagcgcctgttaccagtgctgtgccaggcccacgggctcacaccccagcaggtggtggccatcgccagcaacggcggcggcaagcaggccctggaaaccgtgcagaggctgctgcctgtgctgtgccaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtctggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 34 - DNA, 7858 bp, Artificial sequence
  pCAG-RabChtTal-2
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaagttcgttccaccgtcgctcagcaccatgaagcactggtggggcacggtttcacacacgcccatattgtggctctgtctcaacatcccgctgcactcgggactgtggccgtcaaatatcaggacatgatcgccgctctgcctgaggcaacccacgaagccattgttggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttacaccccagcaggtggtggccattgctagcaacaatgggggcaagcaggctctggagacagtgcagcgcctgctgcctgtgctgtgccaggctcatggcctcacacctcagcaggtcgtcgccattgcttctaacaatggagggaagcaggctctggagactgtgcagagactgctgccagtgctgtgccaggcccatggcctaacaccccagcaggtggtggccatcgccagccacgacggcggcaagcaggccctggaaaccgtgcagaggctgctgcctgtgctgtgccaggctcatggcctgacacctcagcaggtcgtcgccatcgccagccacgacggcggcaagcaggccctggaaaccgtgcagaggctgctgccagtgctgtgccaggcccatggcttaacaccccagcaggtggtggccatcgctagtcatgacgggggcaaacaggctctggaaacagtacagcggctgttacctgtgctgtgccaggctcatggcttgacacctcagcaggtcgtcgctatcgcctctaataatgggggcaagcaggctctggagacagtacagcgcctgttaccagtgctgtgccaggcccacgggctcacaccccagcaggtggtggcaattgcttccaataacggcggaaaacaggctctggaaaccgtccagaggctgctgcctgtgctgtgccaggctcacggtctaacaccccagcaggtggtggccatcgcttccaacggagggggcaaacaggctctggaaacagtgcagaggctgctgcctgtgctgtgccaggctcatggcctcacacctgagcaggtcgtcgccatcgccagcaacatcggcggcaagcaggccctggaaaccgtgcagaggctgctgccagtgctgtgccaggcccatggcctaacaccccagcaggtggtggcaattgcttccaataacggcggaaaacaggctctggaaaccgtccagaggctgctgcctgtgctgtgccaggctcatggcctgacacctcagcaggtcgtcgcaatcgcctccaatggcggagggaagcaggccctggaaacggtgcagagactgttaccagtgctgtgccaggcccatggcttaacaccccagcaggtggtggcaatcgcctctaataacggagggaagcaggccctggaaaccgtgcagagactgttacctgtgctgtgccaggctcatggcttgacacctcagcaggtcgtcgctatcgctagtcatgatggcggaaaacaggctctggaaactgtgcagcggctgctcccagtgctgtgccaggcccacgggctcacaccccagcaggtggtggcaatcgcctctaataacggagggaagcaggccctggaaaccgtgcagagactgttacctgtgctgtgccaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtctggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 35 - protein, 932 aa, Artificial sequence
  RabChtTal-1
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 36 - protein, 932 aa, Artificial sequence
  RabChtTal-2
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 37 - DNA, 9989 bp, Artificial sequence
  pCMV-Rab-Reporter(hygro)
gaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggacactgcagagacctacttcactaacaaccggtatggtcgccagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtatcaagcgctatgtgcaccaaaacttctcctcgcactaccgggccaccattggtcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaggggatcccccgggctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgagacggaaagaaccagctggggctcgatcctctagagtcgacgtttgatctgatatcatcgatgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatcaaacatgcatgaagttcctattccgaagttcctattctctagaaagtataggaacttcataaaacctgcaggcatgcaagcgatcgcggccggccaaggcccgcggggccactagttctagagcggccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 38 - protein, 34 aa, Artificial sequence
  Tal effector motif (repeat) #11 of the Xanthomonas Hax3 protein
LTPEQVVAIASNIGGKQALETVQRLLPVLCQAHG

SEQ ID NO: 39 - protein, 34 aa, Artificial sequence
  Tal effector motif (repeat) #5 derived from the Xanthomonas Hax3 protein
LTPQQVVAIASHDGGKQALETVQRLLPVLCQAHG

SEQ ID NO: 40 - protein, 34 aa, Artificial sequence
  Tal effector motif (repeat) #4 from the Xanthomonas Hax4 protein
LTPQQVVAIASNGGGKQALETVQRLLPVLCQAHG

SEQ ID NO: 41 - protein, 34 aa, Artificial sequence
  Tal effector motif (repeat) #4 from the Hax4 protein with replacement of the amino acids 12 into N and 13 into N
LTPQQVVAIASNNGGKQALETVQRLLPVLCQAHG

(19) United States
(12) Patent Application Publication
(10) Pub. No.: US 2014/0304847 A1
(43) Pub. Date: Oct. 9, 2014

(54) RECOMBINATION EFFICIENCY BY INHIBITION OF NHEJ DNA REPAIR

(71) Applicants: Ralf Kühn, Freising (DE); Wolfgang Wurst, Munchen (DE)

(72) Inventors: Ralf Kühn, Freising (DE); Wolfgang Wurst, Munchen (DE)

(21) Appl. No.: 14/124,106

(22) PCT Filed: Jun. 6, 2012
(86) PCT No.: PCT/EP2012/060716

§ 371 Date: Jan. 21, 2014

(30) Foreign Application Priority Data
Jun. 7, 2011 (EP) 11004637.2

(51) Int. Cl. C12N 15/85

(52) U.S. Cl. CPC C12N 15/85; C12N 15/8509
USPC 800/21; 435/462

(57) ABSTRACT

The present invention relates to a method for modifying a target sequence in the genome of a mammalian cell, the method comprising the step of introducing into a mammalian cell: a. one or more compounds that introduce double-strand breaks in said target sequence; b. one or more DNA molecules comprising a donor DNA sequence to be incorporated by homologous recombination into the genomic DNA of said mammalian cell within said target sequence, wherein said donor DNA sequence is flanked upstream by a first flanking element and downstream by a second flanking element, wherein said first and second flanking element are different and wherein each of said first and second flanking sequence are homologous to a continuous DNA sequence on either side of the double-strand break introduced by said one or more compounds of a. within said target sequence in the genome of said mammalian cell; and c. one or more compounds that decrease the activity of the non-homologous end joining (NHEJ) DNA repair complex in said mammalian cell. Further, the invention relates to a method of producing a non-human mammal carrying a modified target sequence in its genome.

The present invention relates to a method for modifying a target sequence in the genome of a mammalian cell, the method comprising the step of introducing into a mammalian cell: a. one or more compounds that introduce double-strand breaks in said target sequence; b. one or more DNA molecules comprising a donor DNA sequence to be incorporated by homologous recombination into the genomic DNA of said mammalian cell within said target sequence, wherein said donor DNA sequence is flanked upstream by a first flanking element and downstream by a second flanking element, wherein said first and second flanking element are different and wherein each of said first and second flanking element are homologous to a continuous DNA sequence on either side of the double-strand break introduced by said one or more compounds of a. within said target sequence in the genome of said mammalian cell; and c. one or more compounds that decrease the activity of the non-homologous end joining (NHEJ) DNA repair complex in said mammalian cell. Further, the invention relates to a method of producing a non-human mammal carrying a modified target sequence in its genome.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Pluripotent ES cell lines are routinely used to generate knockout mice for biological and medical research. The production of knockout mice requires the modification of a target gene in ES cells by homologous recombination (HR), the selection of recombined ES cells and the subsequent generation of chimaeric mice that transmit the targeted allele to their offspring (1). HR in ES cells is a rare event found at an absolute frequency of 10⁻⁵-10⁻⁷ of transfected cells such that large number of cells and drug selection are required to isolate recombined ES cell clones. Altogether this procedure is time consuming and labor intense but could not be replaced so far by more straightforward and simple protocols.

Fertilised mammalian oocytes (zygotes) represent a logical substrate for the direct generation of genetically modified animals since the entire organism is derived from this one-cell embryo. The direct manipulation of the mouse genome in zygotes could reduce the time to obtain targeted mutants and avoid the use of selection markers. Moreover, gene targeting in zygotes provides an ES cell independent paradigm to manipulate the genome of mammals and any other vertebrate. However, while the injection of DNA fragments into a zygotic pronucleus is a routine procedure to produce transgenic mice or rats by random integration (2), it is not established to obtain HR events in the pronucleus. An early report on such an attempt showed that HR can occur in the murine pronucleus but the frequency of recombination was below 0.1% (3) making this an impractical procedure due to the limitation in producing and handling large numbers of mouse zygotes.

Zinc-finger nucleases (ZFN) link a DNA binding domain of the zinc-finger type to the nuclease domain of, e.g., FokI and enable the induction of double-strand breaks (DSB) at preselected genomic sites (4). DSBs closed by the error-prone, non-homologous end-joining (NHEJ) DNA repair pathway frequently exhibit nucleotide deletions and insertions at the cleavage site. This technology has been applied to introduce knockout mutations into the germline of rats and zebrafish by the expression of ZFNs in early embryos that target coding sequences (5, 6). Using the yeast homing endonuclease I-SceI it has been initially shown that the induction of DSBs at genomic insertions of I-SceI recognition sites increases the rate of HR in mammalian cells by several orders of magnitude (7). Artificially designed ZFNs further increased the ability to generate site-specific double-strand breaks in endogenous genes, without the requirement to introduce artificial nuclease recognition sites. Following this principle ZFNs have been used to achieve efficient HR of gene targeting vectors with various endogenous loci in cultured and primary mammalian cells (4, 8). Recently, the zinc-finger DNA binding domain could be also replaced by the DNA binding domain of TAL effector proteins (9).

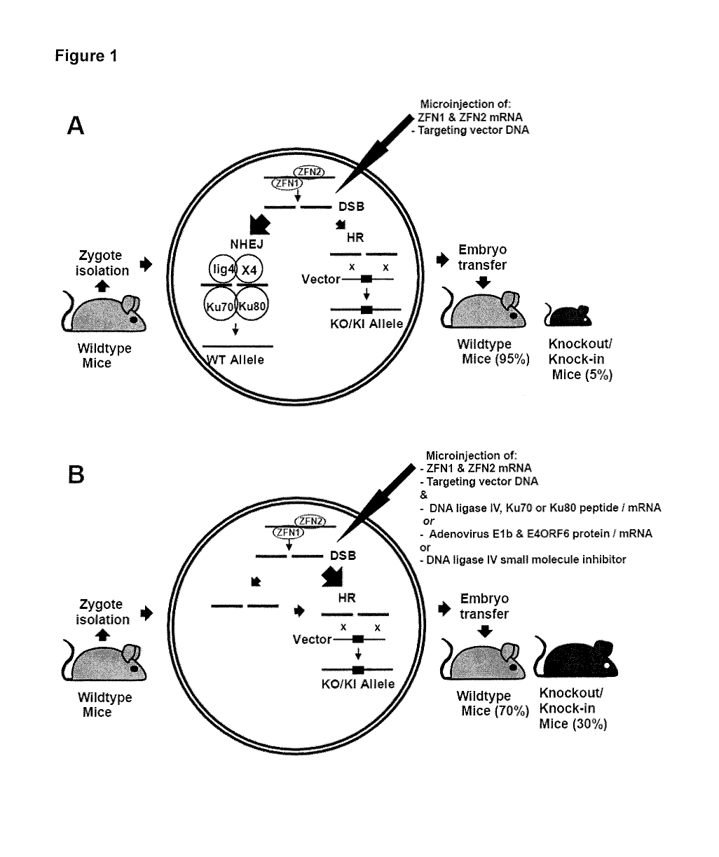

The ZFN technology has been further demonstrated to improve the frequency of HR in mouse zygotes by the expression of target gene specific ZFNs (FIG. 1A). By coinjection of mRNA for ZFNs and specific gene targeting vectors into one pronucleus of mouse or rat zygotes, targeted mutants were obtained with a frequency of up to ˜5% ((10-12). However, the analysis of the non-targeted animals derived from such experiments showed that DSBs, that lead to NHEJ-induced loss of nucleotides, occurred in the target gene loci of injected zygotes at a much higher rate of 20-30%, (6, 11-13). Presumably, the majority of ZFN induced DSBs are rapidly closed by the enzymatic machinery of the NHEJ DNA repair pathway (14) before HR with an introduced targeting vector can occur. It follows from the above that it is desirable to increase the rate of homologous recombination events so as to improve the overall procedure of modifying the genome of a target cell.

The technical problem underlying the present invention was to identify alternative and/or improved means and methods to modifying a target sequence in the genome of a mammalian cell.

The solution to this technical problem is achieved by providing the embodiments characterized in the claims.

Accordingly, the present invention relates in a first embodiment to a method for modifying a target sequence in the genome of a mammalian cell, the method comprising the step of introducing into a mammalian cell: a. one or more compounds that introduce double-strand breaks in said target sequence; b. one or more DNA molecules comprising a donor DNA sequence to be incorporated by homologous recombination into the genomic DNA of said mammalian cell within said target sequence, wherein said donor DNA sequence is flanked upstream by a first flanking element and downstream by a second flanking element, wherein said first and second flanking element are different and wherein each of said first and second flanking element are homologous to a continuous DNA sequence on either side of the double-strand break introduced by said one or more compounds of a. within said target sequence in the genome of said mammalian cell; and c. one or more compounds that decrease the activity of the non-homologous end joining (NHEJ) DNA repair complex in said mammalian cell.

The term “modifying” as used in accordance with the present invention refers to site-specific genomic manipulations resulting in changes in the nucleotide sequence. The genetic material comprising these changes in its nucleotide sequence is also referred to herein as the “modified target sequence”. The term “modifying” includes, but is not limited to, substitution, insertion and deletion of one or more nucleotides within the target sequence. In the process of homologous recombination, the end product may reflect a deletion of sequences. As is understood by the skilled person, a homologous recombination, on the other hand, always also includes the incorporation of genetic material from the donor DNA sequence, which in this embodiment, however, leads to an overall deletion.

The term “substitution”, as used herein, refers to the replacement of nucleotides with other nucleotides. The term includes for example the replacement of single nucleotides resulting in point mutations. Said point mutations can lead to an amino acid exchange in the resulting protein product but may also not be reflected on the amino acid level. Also encompassed by the term “substitution” are mutations resulting in the replacement of multiple nucleotides, such as for example parts of genes, such as parts of exons or introns as well as replacement of entire genes.

The term “insertion” in accordance with the present invention refers to the incorporation of one or more nucleotides into a nucleic acid molecule. Insertion of parts of genes, such as parts of exons or introns as well as insertion of entire genes is also encompassed by the term “insertion”. When the number of inserted nucleotides is not dividable by three, the insertion can result in a frameshift mutation within a coding sequence of a gene. Such frameshift mutations will alter the amino acids encoded by a gene following the mutation. In some cases, such a mutation will cause the active translation of the gene to encounter a premature stop codon, resulting in an end to translation and the production of a truncated protein. When the number of inserted nucleotides is instead dividable by three, the resulting insertion is an “in-frame insertion”. In this case, the reading frame remains intact after the insertion and translation will most likely run to completion if the inserted nucleotides do not code for a stop codon. However, because of the inserted nucleotides, the resulting protein will contain, depending on the size of the insertion, one or multiple new amino acids that may effect the function of the protein.

The term “deletion” as used in accordance with the present invention refers to the loss of nucleotides or part of genes, such as exons or introns as well as entire genes. As defined with regard to the term “insertion”, the deletion of a number of nucleotides that is not evenly dividable by three will lead to a frameshift mutation, causing all of the codons occurring after the deletion to be read incorrectly during translation, potentially producing a severely altered and most likely non-functional protein. If a deletion does not result in a frameshift mutation, i.e. because the number of nucleotides deleted is dividable by three, the resulting protein is nonetheless altered as the it will lack, depending on the size of the deletion, several amino acids that may affect or effect the function of the protein.

The above defined modifications are not restricted to coding regions in the genome, but can also occur in non-coding regions of the target genome, for example in regulatory regions such as promoter or enhancer elements or in introns.

Examples of modifications of the target genome include, without being limiting, the introduction of mutations into a wild type gene in order to analyse its effect on gene function; the replacement of an entire gene with a mutated gene or, alternatively, if the target sequence comprises mutation(s), the alteration of these mutations to identify which mutation is causative of a particular effect; the removal of entire genes or proteins or the removal of regulatory elements from genes or proteins as well as the introduction of fusion-partners, such as for example purification tags such as the his-tag or the tap-tag etc. In the latter case, the term “addition” may also be used instead of “insertion” so as to describe the preferable addition of a tag to a terminus of a polypeptide rather than within the sequence of a polypeptide

In accordance with the present invention, the term “target sequence in the genome” refers to the genomic location that is to be modified by the method of the invention. The “target sequence in the genome” comprises but is not restricted to the nucleotide(s) subject to the particular modification. Furthermore, the term “target sequence in the genome” also comprises regions for binding of homologous sequences of a second nucleic acid molecule. In other words, the term “target sequence in the genome” also comprises the sequence flanking/surrounding the relevant nucleotide(s) to be modified. In some instances, the term “target sequence” may also refer to the entire gene to be modified.

The term “mammalian cell” as used herein, is well known in the art and refers to any cell belonging to an animal that is grouped into the class of mammalia. The term “cell” as used herein can refer to a single and/or isolated cell or to a cell that is part of a multicellular entity such as a tissue, an organism or a cell culture another. In other words the method can be performed in vivo, ex vivo or in vitro. Depending on the particular goal to be achieved through modifying the genome of a mammalian cell, cells of different mammalian subclasses such as prototheria or theria may be used. For example, within the subclass of theria, preferably cells of animals of the infraclass eutheria, more preferably of the order primates, artiodactyla, perissodactyla, rodentia and lagomorpha are used in the method of the invention. Furthermore, within a species one may choose a cell to be used in the method of the invention based on the tissue type and/or capacity to differentiate equally depending on the goal to be achieved by modifying the genome. Three basic categories of cells make up the mammalian body: germ cells, somatic cells and stem cells. A germ cell is a cell that gives rise to gametes and thus is continuous through the generations. Stem cells can divide and differentiate into diverse specialized cell types as well as self renew to produce more stem cells. In mammals there are two main types of stem cells: embryonic stem cells and adult stem cells. Somatic cells include all cells that are not a gametes, gametocytes or undifferentiated stem cells. The cells of a mammal can also be grouped by their ability to differentiate. A totipotent (also known as omnipotent) cell is a cell that is able to differentiate into all cell types of an adult organism including placental tissue such as a zygote (fertilized oocyte) and subsequent blastomeres, whereas pluripotent cells, such as embryonic stem cells, cannot contribute to extraembryonic tissue such as the placenta, but have the potential to differentiate into any of the three germ layers endoderm, mesoderm and ectoderm. Multipotent progenitor cells have the potential to give rise to cells from multiple, but limited number of cell lineages. Further, there are oligopotent cells that can develop into only a few cell types and unipotent cells (also sometimes termed a precursor cell) that can develop into only one cell type. There are four basic types of tissues: muscle tissue, nervous tissue, connective tissue and epithelial tissue that a cell to be used in the method of the invention can be derived from, such as for example hematopoietic stem cells or neuronal stem cells. To the extent human cells are envisaged for use in the method of the invention, it is preferred that such human cell is not obtained from a human embryo, in particular not via methods entailing destruction of a human embryo. On the other hand, human embryonic stem cells are at the skilled person's disposal such as taken from existent embryonic stem cell lines commercially available. Accordingly, the present invention may be worked with human embryonic stem cells without any need to use or destroy a human embryo. Alternatively, or instead of human embryonic stem cells, pluripotent cells that resemble embryonic stem cells such induced pluripotent stem (iPS) cells may be used, the generation of which is state of the art (Hargus G et al., Proc Natl Acad Sci USA 107:15921-15926; Jaenisch R. and Young R., 2008, Cell 132:567-582; Saha K, and Jaenisch R., 2009, Cell Stem Cell 5:584-595.

The skilled person is well aware of “compounds that introduce double-strand breaks” in said target sequence. Any compound may be used as long as it does not compromise cell viability and is target sequence specific, optionally when applied in suitable amounts/concentrations. Target sequence specificity means in accordance with the method of the invention that a double-strand break is exclusively introduced at the target sequence and not elsewhere in the genome to be modified. For example, restriction nucleases are well-known to introduce double-strand breaks into genomic DNA and are described herein below. In combination with a DNA-targeting molecule, this capability allows to introduce site-specific strand breaks. As an example, zinc-finger nucleases represent such combination in the form of fusion (poly)peptides. The term “one or more” as used herein may refer to the same or different compounds. In the case of the compounds being different, they may exhibit the same specificity as regards the target sequence or they may target a further target sequence. In other words, the method of the invention can be adapted to enable simultaneous modification of two or more different target sequences in the genome at one time. Accordingly, the simultaneous modification of at least two, at least three, at least four and at least five, such as, e.g., at least six or at least seven target sequences is envisioned.

Said one or more compounds, if (poly)peptides, may also be introduced into a mammalian cell in the form of an expressible nucleic acid molecule encoding said one or more compounds. Preferably, but without limitation, said nucleic acid molecule is an mRNA molecule. Also, said nucleic acid molecule may be a DNA molecule which, e.g., may further be comprised in a DNA molecule comprising a donor DNA sequence and a first and second flanking element according to b. of the method of the invention.

The term “homologous recombination”, is used according to the definitions provided in the art. Thus, it refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbiol Mol Biol Rev 1999; 63:349-404). In the method of the present invention, homologous recombination is enabled by the presence of said first and said second flanking element being placed upstream (5′) and downstream (3′), respectively, of said donor DNA sequence each of which being homologous to a continuous DNA sequence within said target sequence.

In accordance with the present invention, the term “donor DNA sequence” refers to a DNA sequence that serves as a template in the process of homologous recombination and that carries the modification that is to be introduced into the target sequence. By using this donor DNA sequence as a template, the genetic information, including the modifications, is copied into the target sequence within the genome of the cell by way of homologous recombination. In non-limiting examples, the donor nucleic acid sequence can be essentially identical to the part of the target sequence to be replaced, with the exception of one nucleotide which differs and results in the introduction of a point mutation upon homologous recombination or it can consist of an additional gene previously not present in the target sequence. Conceivably, the nature, i.e. its length, base composition, similarity with the target sequence, of the donor DNA sequence depends on how the target sequence is to be modified as well as the particular goal to be achieved by the modification of the target sequence. It is understood by those skilled in the art that said donor DNA sequence is flanked by sequences that are homologous to sequences within the target sequence to enable homologous recombination to take place leading to the incorporation of the donor DNA sequence into the genome of said mammalian cell. In addition to being homologous to a continuous DNA sequence within the genomic DNA, the first and the second flanking element are different to allow targeted homologous recombination to take place.

The term “homologous to a continuous DNA sequence on either side of the double-strand break introduced by said one or more compounds of a. within said target sequence”, in accordance with the present invention, refers to regions having sufficient sequence identity to ensure specific binding to the target sequences that lie upstream and downstream of the location of the double-strand break. The term “homologous” as used herein can be interchanged with the term “identical” as outlined below with regard to varying levels of sequence identity. Methods to evaluate the identity level between two nucleic acid sequences are well known in the art. For example, the sequences can be aligned electronically using suitable computer programs known in the art. Such programs comprise BLAST (Altschul et al. (1990) J. Mol. Biol. 215, 403), variants thereof such as WU-BLAST (Altschul and Gish (1996) Methods Enzymol. 266, 460), FASTA (Pearson and Lipman (1988) Proc. Natl. Acad. Sci. USA 85, 2444) or implementations of the Smith-Waterman algorithm (SSEARCH, Smith and Waterman (1981) J. Mol. Biol., 147, 195). These programs, in addition to providing a pairwise sequence alignment, also report the sequence identity level (usually in percent identity) and the probability for the occurrence of the alignment by chance (P-value) and can further be used to predict the occurrence of specific binding. Preferably, the program BLAST is used in accordance with the invention.

Preferably, said first and second flanking element being “homologous to a continuous DNA sequence within said target sequence” (also referred to as “homology arms” in the art) have a sequence identity with the corresponding part of the target sequence of at least 95%, more preferred at least 97%, more preferred at least 98%, more preferred at least 99%, even more preferred at least 99.9% and most preferred 100%. The above defined sequence identities are defined only with respect to those parts of the target sequence which serve as binding sites for the homology arms, i.e. said first and said second flanking element. Thus, the overall sequence identity between the entire target sequence and the homologous regions of the nucleic acid molecule of step (b) of the method of modifying a target sequence of the present invention can differ from the above defined sequence identities, due to the presence of the part of the target sequence which is to be replaced by the donor DNA sequence.

The flanking elements homologous to the target sequence comprised in the DNA molecule have a length of at least 200 bp each. Preferably, the elements each have a length of at least 250 nucleotides, at least 300 nucleotides, at least 400 nucleotides, at least 500 nucleotides, such as at least 600 nucleotides, at least 750 bp nucleotides, more preferably at least 1000 nucleotides, such as at least 1500 nucleotides, even more preferably at least 2000 nucleotides and most preferably at least 2500 nucleotides. The maximum length of the elements homologous to the target sequence comprised in the nucleic acid molecule depends on the type of cloning vector used and can be up to a length 20.000 nucleotides each in E. coli high copy plasmids using the col EI replication origin (e.g. pBluescript) or up to a length of 300.000 nucleotides each in plasmids using the F-factor origin (e.g. in BAC vectors such as for example pTARBAC1).

In accordance with the method of modifying a target sequence of the present invention, the one or more DNA molecules introduced into the cell in b. comprise the donor DNA sequence as defined above as well as the flanking elements that are homologous to the target sequence and flank the donor DNA sequence. In line with the interpretation of the term “one or more” above, the DNA molecules may comprise or consist of the same donor DNA sequence and flanking elements or may comprise or consist of different donor DNA sequences and flanking elements. In other words, the method of the invention also enables simultaneous modification of at least two or more target sequences, as outlined above. The DNA molecules may take the form of a circular double-stranded DNA molecule or alternatively exist in non-circular form. Circular DNA molecules may comprise further sequence elements that the donor DNA sequence and the first and second flanking element of b, i.e. the latter sequences may be inserted into several commercially available vectors. However, because the DNA molecules are to serve as templates for homologous recombination, the person skilled in the art understands that a vector to be used in the method of the invention, may, preferably, only contain the sequences necessary for serving as template and, e.g., a multiple cloning site. Should the DNA molecule of b. also comprise sequences encoding the one or more compounds in a. and/or c. (as outlined below) a corresponding vector will comprise further elements allowing expression of said one or more compounds in a mammalian cell. The skilled person is in the position to determine the sequences necessary to achieve expression in a mammalian cell or can use one of the various expression vectors available in the art.

The DNA molecules comprising the donor DNA sequence and the flanking elements are—necessarily if the nuclease binding site is contained undisrupted within one of the flanking elements and preferably if the nuclease binding site is disrupted by the donor sequence, i.e. one part on each of the flanking elements—modified so that the one or more compounds of a. do not introduce a double-strand break into the sequence of the donor DNA as part of a DNA molecule. When the compound is a TAL or zinc-finger nuclease or an engineered meganuclease, this can be achieved, e.g., by modifying either the binding or cleavage motif (see example, introduction of “silent mutation”).

The term “non-homologous end joining (NHEJ) DNA repair complex” as used in accordance with the method of the invention corresponds to the meaning known in the art. Chromosomal DSBs (double-strand breaks) can result from either endogenous or exogenous sources. Naturally occurring DSBs are generated spontaneously during DNA synthesis when the replication fork encounters a damaged template and during certain specialized cellular processes, including V(D)J recombination, class-switch recombination at the immunoglobulin heavy chain (IgH) locus and meiosis. In addition, exposure of cells to ionizing radiation (X-rays and gamma rays), UV light, topoisomerase poisons or radiomimetic drugs can produce DSBs. The NHEJ (non-homologous end-joining) pathway joins the two ends of a DSB through a process largely independent of homology. Depending on the specific sequences and chemical modifications generated at the DSB, NHEJ may be precise or mutagenic. For NHEJ-mediated repair, the Ku70/Ku80 (Ku) proteins bind with high affinity to DNA termini in a structure-specific manner and can promote end alignment of the two DNA ends. The DNA-bound Ku heterodimer recruits DNA-PK_{cs} (DNA-dependent protein kinase catalytic subunit), and activates its kinase function. Together with the Artemis protein, DNA-PK_{cs} can stimulate processing of the DNA ends. Finally, the XRCC4 (X-ray repair complementing defective repair in Chinese hamster cells 4)—DNA ligase IV complex, which does not form a stable complex with DNA but interacts stably with the Ku—DNA complex, carries out the ligation step to complete repair. Ligation is central to DSB repair by the NHEJ pathway and requires the concerted action of DNA Ligase-IV, XRCC4, and Cer-XLF. In vivo, DNA Ligase-IV associates tightly with XRCC4 that serves as a multipurpose partner for Ligase-IV, not only stimulating its adenylation and promoting stable interactions with DNA, but also protecting it from degradation. The stoichiometric ratio of the XRCC4/Ligase-IV complex is 2:1. Within the XRCC4/Ligase-IV complex, interactions have been mapped to the central coiled-coil domain of XRCC4 and to the inter-BRCT (BRCA1 [breast cancer associated 1] C terminal) domain linker at the C terminus of DNA-Ligase-IV. This XRCC4-interacting region (XIR) of Ligase-IV appears necessary and sufficient for XRCC4/Ligase-IV interaction (Lieber M R. The mechanism of double-strand DNA break repair by the nonhomologous DNA end-joining pathway. Annu Rev Biochem 79:181-211).

The “one or more compounds that decrease the activity of the NHEJ DNA repair complex” can be any compounds that directly or indirectly affect the activity of the NHEJ DNA repair complex. Preferably, said compounds do not affect the viability of a mammalian cell. The compounds may be chosen from organic or anorganic compounds. Also, antibodies, peptides, RNAi molecules and/or small molecules as outlined herein below in detail are envisioned. The compounds may target any component of the NHEJ DNA repair complex. For example, the DNA ligase IV may be a target, or Ku70 or Ku80 as explained below. The term “activity of the NHEJ DNA repair complex” refers in accordance with the invention to the capability of the NHEJ DNA repair complex to repair double-strand breaks as outlined above. Hence, a “decrease in the activity of the NHEJ DNA repair complex” may mean a decrease in the rate of double-strand break repairs per a defined period of time in comparison to a suitable control or the occurrence of no repair events at all. For example, a decrease in the activity of NHEJ DNA repair complex includes a decrease in the rate of double-strand break repairs of at least (for each value) 5%, 10%, 15%, 20%, 25%, 30%, 35%, 40%, 45%, 50%, more preferred of at least 55%, 60%, 65%, 70% such as 75%, 80%, 85% and even more preferred of at least 90%, 91%, 92%, 93%, 94%, 95%, 96%, 97%, 98% or at least 99% and most preferred of 100%, i.e. no strand break repair event occurs, as compared to a suitable control. Said control can be, for example, an NHEJ DNA repair complex whose activity is assessed in the absence of said one or more compounds that decrease the activity of said NHEJ DNA repair complex. Preferably, the control is an unmanipulated, i.e. an endogenously occurring non-mutant NHEJ DNA repair complex. Preferred is that the activity of a control is assessed more than once or that several samples of said control are obtained in order to increase the reliability of the data relating to the activity. The data may further be pooled to calculate the mean or median and optionally the variance for each control. Furthermore preferred is that the activity read-outs are compared to the activity read-outs in samples of more than one control such as at least 2, 10, 20 or more preferred 50, and most preferred 100 and more controls. Furthermore, also preferred is that the activity read-outs of the samples of the controls are pooled and the mean or median and optionally the variance is calculated. These values may, e.g., be deposited into a database as a standardized value for the activity of an NHEJ DNA repair complex and if required retrieved from a database, hence making the need to also experimentally assess the activity levels in a control sample dispensable. Accordingly a control may also be a database entry. Moreover, by using the variance of the expression level of the control sample, the statistical significance of deviations from the mean of controls in the sample to be assessed may be determined. The skilled person is in the position to assess and determine a decrease in the activity of the NHEJ DNA repair complex with standard experiments. This can be achieved, e.g., by using appropriate reporter gene constructs such as for example described in Mao Z. et al., 2008, DNA Repair (Amst) 7:1765-1771; Seluanov A. et al., J V is Exp, 2010, September 8; (43); Weinstock D M. Et al., 2006, Methods Enzymol 409:524-540) In accordance with the method of the invention, it is understood that the decrease in the activity of the NHEJ DNA repair complex by the one or more compounds is not caused by a change in the genomic DNA sequence of the lig4-gene.

In accordance with the method of the present invention, the introduction of the one or more compounds of a., the one or more nucleic acid molecules of b. and the one or more compounds of c. into the mammalian cell can be carried out concomitantly, i.e. at the same time or can be carried out separately, i.e. individually and at different time points. When the introduction is carried out concomitantly, the one or more compounds of a., the one or more nucleic acid molecules of b. and the one or more compounds of c. can be administered in parallel, for example using three separate injection needles or can be mixed together and, for example, be injected using one needle. Preferably, in the case of separate introduction, the one or more compounds of a. and the one or more compounds of c are administered together, while only the one or more DNA molecules of b. are administered at a different point of time. In this case, the one or more compounds of a. and the one or more compounds of c. are preferably administered prior to the administration of the DNA molecules of b. Generally, in the case of separate administration, the differing time points of administration should be selected so that the one or more compounds of a., the one or more nucleic acid molecules of b. and the one or more compounds of c. are administered can still act in concert to achieve modification of a target sequence in accordance with the method of the invention. The person skilled in the art is in the position to determine corresponding time points by conducting experiments that compare modification efficiency at different time points.

It will be appreciated by one of skill in the art that said one or more DNA molecules to be introduced into the cell in b. may comprise all a nucleic acid molecule (sequence) encoding said one or more compounds introducing double-strand breaks, the nucleic acid molecule comprising the donor nucleic acid sequence and the flanking elements homologous to the target sequence, and the a nucleic acid molecule (sequence) encoding said one or more compounds decreasing the activity of the NHEJ DNA repair complex. Alternatively, the nucleic acid molecule of b. may be a distinct nucleic acid molecule, to be introduced in addition to the nucleic acid molecules encoding said one or more compounds of a and/or c.

Introduction into a mammalian cell of the compounds of a. and c. as well as of the nucleic acid molecules of b. can be achieved by methods known in the art and depends on the nature of said compounds or nucleic acid molecules. For example, and in the case of introducing nucleic acid molecules, said introducing can be achieved by chemical based methods (calcium phosphate, liposomes, DEAE-dextrane, polyethylenimine, nucleofection), non chemical methods (electroporation, sonoporation, optical transfection, gene electrotransfer, hydrodynamic delivery), particle-based methods (gene gun, magnetofection, impalefection) and viral methods. Preferably, the nucleic acid molecules are to be introduced into the nucleus by methods such as, e.g., microinjection or nucleofection. Methods for carrying out microinjection are well known in the art and are described for example in Nagy et al. (Nagy A, Gertsenstein M, Vintersten K, Behringer R., 2003. Manipulating the Mouse Embryo. Cold Spring Harbour, New York: Cold Spring Harbour Laboratory Press) as well as in the examples herein below. It is understood by the skilled person that depending on the method of introduction it may be advantageous to adapt the DNA molecules of b. For example, a linear DNA molecule may be more efficient in HR when using electroporation as method to introduce said DNA molecule into a mammalian cell, whereas a circular DNA molecule may be more advantageous when injecting cells.

Also envisaged in the context of the method of the invention is that the mammalian cells are analysed for successful modification of the target genome.

Methods for analysing for the presence or absence of a modification are well known in the art and include, without being limiting, assays based on physical separation of nucleic acid molecules, sequencing assays as well as cleavage and digestion assays and DNA analysis by the polymerase chain reaction (PCR).

Examples for assays based on physical separation of nucleic acid molecules include without limitation MALDI-TOF, denaturating gradient gel electrophoresis and other such methods known in the art, see for example Petersen et al., Hum. Mutat. 20 (2002) 253-259; Hsia et al., Theor. Appl. Genet. 111 (2005) 218-225; Tost and Gut, Clin. Biochem. 35 (2005) 335-350; Palais et al., Anal. Biochem. 346 (2005) 167-175.

Examples for sequencing assays comprise without limitation approaches of sequence analysis by direct sequencing, fluorescent SSCP in an automated DNA sequencer and Pyrosequencing. These procedures are common in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003) 9; Meng et al., J. Clin. Endocrinol. Metab. 90 (2005) 3419-3422.

Examples for cleavage and digestion assays include without limitation restriction digestion assays such as restriction fragments length polymorphism assays (RFLP assays), RNase protection assays, assays based on chemical cleavage methods and enzyme mismatch cleavage assays, see e.g. Youil et al., Proc. Natl. Acad. Sci. U.S.A. 92 (1995) 87-91; Todd et al., J. Oral Maxil. Surg. 59 (2001) 660-667; Amar et al., J. Clin. Microbiol. 40 (2002) 446-452.

Alternatively, instead of analysing the cells for the presence or absence of the desired modification, successfully modified cells may be selected by incorporation of appropriate selection markers. Selection markers include positive and negative selection markers, which are well known in the art and routinely employed by the skilled person. Non-limiting examples of selection markers include dhfr, gpt, neomycin, hygromycin, dihydrofolate reductase, G418 or glutamine synthase (GS) (Murphy et al., Biochem J. 1991, 227:277; Bebbington et al., Bio/Technology 1992, 10:169). Using these markers, the cells are grown in selective medium and the cells with the highest resistance are selected. Also envisaged are combined positive-negative selection markers, which may be incorporated into the target genome by homologous recombination or random integration. After positive selection, the first cassette comprising the positive selection marker flanked by recombinase recognition sites is exchanged by recombinase mediated cassette exchange against a second, marker-less cassette. Clones containing the desired exchange cassette are then obtained by negative selection.

As evident from the example, the inventors have been able to significantly improve the rate at which the genome of mammalian cells can be modified when relying on HR as means to introduce modification into the genome of a mammalian cell. To this end, the present invention relies on the finding that decreasing the activity of the NHEJ DNA repair complex leads to an increase in the rate of homologous recombination occurring subsequent to introducing double-strand breaks and donor DNA sequences in mammalian cells.

While one report indicates that the suppression of NHEJ DNA repair by genetic DNA ligase IV deficiency in Drosophila embryos leads to the preferential resolution of ZFN-induced DSBs with a gene targeting vector by homologous recombination, resulting in the increased recovery of targeted allele (15), other reports suggest that simply removing DNA ligase IV does not necessarily and always reduce the activity of the NHEJ DNA repair complex (McVey et al., Genetics, 168:2067-2076 (2004); Romeijn et al., Genetics, 169:795-806 (2005); Johnson-Schlitz et al., PLoS Genet, 3:e50 (2007); Wei D S, Rong Y S, Genetics, 177:63-77 (2007)). It appears that only removal of DNA ligase IV by way of generating lig4⁻ drosophila embryos which have been shown to be viable and fertile despite the complete absence of Lig4 could effect the increase in homologous recombination events in drosophila. Exemplifying the incomplete understanding of the NHEJ DNA repair complex in development and adulthood in drosophila and even more so the significant cross-species differences between mammals and insects, deficiency of DNA ligase IV in mice leads to embryonic lethality (16). Therefore, and evidently, one cannot expect to achieve the same results in mammalian cells as those shown in drosophila, specifically in view of the apparent uncertainty as regards function and effects of NHEJ DNA repair and even more so in view of the impossibility of applying the same methodical approach as in drosophila, i.e. creating lig4-knock out mutants. In mammalian cells DNA ligase IV appears to constitute a key enzyme in NHEJ repair but it is considered to not be required for HR. Nevertheless, the inventors were able to successfully devise a method that increases the stable transfection efficiency in mammalian cells as outlined herein above and below, in particular in the example section. As is appreciated by the skilled person the method of the invention provides a significant enhancement in the practicability and financing of generating transgenic animals. For example, if oocytes, preferably fertilized oocytes, are used as mammalian cells in the method of the invention, the significantly increased rate of homologous recombination results in a greater number of first generation offspring carrying the desired modification in their genome. Therefore, and for the first time, the method of the invention provides a practical alternative to using the more time-consuming and difficult method involving ES cells as starting material for generating transgenic animals. Evidently, however, also when using ES cells the method of the invention is advantageous. Furthermore, primary fibroblasts or mesenchymal cells can be used for gene targeting and the generation of genetically modified mammals (e.g. sheeps, pigs, etc) by subsequent nuclear transfer. The method of the invention provides a practical means to enhance the frequency at which correctly recombined primary cells can be obtained.

In a preferred embodiment of the method of the invention, said one or more compounds in a. are selected from the group consisting of TAL nucleases, zinc-finger nucleases or engineered meganucleases or nucleic acid molecules encoding said TAL nucleases, zinc-finger nucleases or engineered meganucleases in expressible form.

The term “TAL nuclease” as used herein, is well known in the art and refers to a fusion (poly)peptide comprising a DNA-binding domain, wherein the DNA-binding domain comprises or consists of Tal effector motifs of a TAL effector protein and the non-specific cleavage domain of a restriction nuclease. The fusion (poly)peptide employed in the method of the invention retains or essentially retains the enzymatic activity of the native (restriction) endonuclease. In accordance with the present invention, (restriction) endonuclease function is essentially retained if at least 60% of the biological activity of the endonuclease activity are retained. Preferably, at least 75% or at least 80% of the endonuclease activity are retained. More preferred is that at least 90% such as at least 95%, even more preferred at least 98% such as at least 99% of the biological activity of the endonuclease are retained. Most preferred is that the biological activity is fully, i.e. to 100%, retained. Also in accordance with the invention, fusion (poly)peptides having an increased biological activity compared to the endogenous endonuclease, i.e. more than 100% activity. Methods of assessing biological activity of (restriction) endonucleases are well known to the person skilled in the art and include, without being limiting, the incubation of an endonuclease with recombinant DNA and the analysis of the reaction products by gel electrophoresis (Bloch K D.; Curr Protoc Mol Biol 2001; Chapter 3:Unit 3.2).

The term “Tal effector protein”, as used herein, refers to proteins belonging to the TAL (transcription activator-like) family of proteins. These proteins are expressed by bacterial plant pathogens of the genus Xanthomonas. Members of the large TAL effector family are key virulence factors of Xanthomonas and reprogram host cells by mimicking eukaryotic transcription factors. The pathogenicity of many bacteria depends on the injection of effector proteins via type III secretion into eukaryotic cells in order to manipulate cellular processes. TAL effector proteins from plant pathogenic Xanthomonas are important virulence factors that act as transcriptional activators in the plant cell nucleus. PthXo1, a TAL effector protein of a Xanthomonas rice pathogen, activates expression of the rice gene Os8N3, allowing Xanthomonas to colonize rice plants. TAL effector proteins are characterized by a central domain of tandem repeats, i.e. a DNA-binding domain as well as nuclear localization signals (NLSs) and an acidic transcriptional activation domain. Members of this effector family are highly conserved and differ mainly in the amino acid sequence of their repeats and in the number of repeats. The number and order of repeats in a TAL effector protein determine its specific activity. These repeats are referred to herein as “TAL effector motifs”. One exemplary member of this effector family, AvrBs3 from Xanthomonas campestris pv. vesicatoria, contains 17.5 repeats and induces expression of UPA (up-regulated by AvrBs3) genes, including the Bs3 resistance gene in pepper plants (Kay, et al. 2005 Mol Plant Microbe Interact 18(8): 838-48; Kay, S, and U. Bonas 2009 Curr Opin Microbiol 12(1): 37-43). The repeats of AvrBs3 are essential for DNA binding of AvrBs3 and represent a distinct type of DNA binding domain. The mechanism of sequence specific DNA recognition has been elucidated by recent studies on the AvrBs3, Hax2, Hax3 and Hax4 proteins that revealed the TAL effectors' DNA recognition code (Boch, J., et al. 2009 Science 326: 1509-12).

Tal effector motifs or repeats are 32 to 34 amino acid protein sequence motifs. The amino acid sequences of the repeats are conserved, except for two adjacent highly variable residues (at positions 12 and 13) that determine specificity towards the DNA base A, G, C or T. In other words, binding to DNA is mediated by contacting a nucleotide of the DNA double helix with the variable residues at position 12 and 13 within the Tal effector motif of a particular Tal effector protein (Boch, J., et al. 2009 Science 326: 1509-12). Therefore, a one-to-one correspondence between sequential amino acid repeats in the Tal effector proteins and sequential nucleotides in the target DNA was found. Each Tal effector motif primarily recognizes a single nucleotide within the DNA substrate. For example, the combination of histidine at position 12 and aspartic acid at position 13 specifically binds cytosine; the combination of asparagine at both position 12 and position 13 specifically binds guanosine; the combination of asparagine at position 12 and isoleucine at position 13 specifically binds adenosine and the combination of asparagine at position 12 and glycine at position 13 specifically binds thymidine. Binding to longer DNA sequences is achieved by linking several of these Tal effector motifs in tandem to form a “DNA-binding domain of a Tal effector protein”. Thus, a DNA-binding domain of a Tal effector protein relates to DNA-binding domains found in naturally occurring Tal effector proteins as well as to DNA-binding domains designed to bind to a specific target nucleotide sequence as described in the examples below. The use of such DNA-binding domains of Tal effector proteins for the creation of Tal effector motif—nuclease fusion (poly)peptides that recognize and cleave a specific target sequence depends on the reliable creation of DNA-binding domains of Tal effector proteins that can specifically recognize said particular target. Methods for the generation of DNA-binding domains of Tal effector proteins are disclosed in the appended examples of this application.

Preferably, the DNA-binding domain is derived from the Tal effector motifs found in naturally occurring Tal effector proteins, such as for example Tal effector proteins selected from the group consisting of AvrBs3, Hax2, Hax3 or Hax4 (Bonas et al. 1989. Mol Gen Genet. 218(1): 127-36; Kay et al. 2005 Mol Plant Microbe Interact 18(8): 838-48).

Preferably, the restriction nuclease is an endonuclease. The terms “endonuclease” and “restriction endonuclease” are used herein according to the well-known definitions provided by the art. Both terms thus refer to enzymes capable of cutting nucleic acids by cleaving the phosphodiester bond within a polynucleotide chain. Preferably, the endonuclease is a type II S restriction endonuclease, such as for example FokI, AlwI, SfaNI, SapI, PleI, NnneAIII, MboII, MlyI, MmeI, HpYAV, HphI, HgaI, FauI, EarI, EciI, BtgZI, CspCI, BspQI, BspMI, BsaXI, BsgI, BseI, BpuEI, BmrI, BcgI, BbvI, BaeI, BbsI, AlwI, or AcuI or a type III restriction endonuclease (e.g. EcoP1I, EcoP15I, HinfIII). Also envisaged herein are meganucleases, such as for example I-SceI. Once the DNA-binding domain (of the fusion (poly)peptide comprising a DNA-binding domain of a Tal effector protein and a nuclease domain) is anchored at the recognition site, a signal is transmitted to the endonuclease domain and cleavage occurs. The distance of the cleavage site to the DNA-binding site of the fusion (poly)peptide depends on the particular endonuclease present in the fusion (poly)peptide. For example, naturally occurring endonucleases such as FokI and EcoP15I cut at 9/13 and 27 bp distance from the DNA binding site, respectively.

Envisaged in accordance with the present invention are fusion (poly)peptides that are provided as functional monomers comprising a DNA-binding domain of a Tal effector protein coupled with a single nuclease domain. The DNA-binding domain of a Tal effector protein and the cleavage domain of the nuclease may be directly fused to one another or may be fused via a linker.

The term “linker” as used in accordance with the present invention relates to a sequel of amino acids (i.e. peptide linkers) as well as to non-peptide linkers.

Peptide linkers as envisaged by the present invention are (poly)peptide linkers of at least 1 amino acid in length. Preferably, the linkers are 1 to 100 amino acids in length. More preferably, the linkers are 5 to 50 amino acids in length and even more preferably, the linkers are 10 to 20 amino acids in length. It is well known to the skilled person that the nature, i.e. the length and/or amino acid sequence of the linker may modify or enhance the stability and/or solubility of the molecule. Thus, the length and sequence of a linker depends on the composition of the respective portions of the fusion (poly)peptide.

The skilled person is aware of methods to test the suitability of different linkers. For example, the properties of the molecule can easily be tested by testing the nuclease activity as well as the DNA-binding specificity of the respective portions of the fusion (poly)peptide to be used in the method of the invention.

It will be appreciated by the skilled person that when the fusion (poly)peptide is provided as a nucleic acid molecule encoding the fusion (poly)peptide in expressible form, the linker is a peptide linker also encoded by said nucleic acid molecule.

The term “non-peptide linker”, as used in accordance with the present invention, refers to linkage groups having two or more reactive groups but excluding peptide linkers as defined above. For example, the non-peptide linker may be a polymer having reactive groups at both ends, which individually bind to reactive groups of the individual portions of the fusion (poly)peptide, for example, an amino terminus, a lysine residue, a histidine residue or a cysteine residue. The reactive groups of the polymer include an aldehyde group, a propionic aldehyde group, a butyl aldehyde group, a maleimide group, a ketone group, a vinyl sulfone group, a thiol group, a hydrazide group, a carbonyldimidazole (CDI) group, a nitrophenyl carbonate (NPC) group, a trysylate group, an isocyanate group, and succinimide derivatives. Examples of succinimide derivatives include succinimidyl propionate (SPA), succinimidyl butanoic acid (SBA), succinimidyl carboxymethylate (SCM), succinimidyl succinamide (SSA), succinimidyl succinate (SS), succinimidyl carbonate, and N-hydroxy succinimide (NHS). The reactive groups at both ends of the non-peptide polymer may be the same or different. For example, the non-peptide polymer may have a maleimide group at one end and an aldehyde group at another end. Preferably, the linker is a peptide linker. More preferably, the peptide linker consists of seven glycine residues.

Preferably, the TAL nuclease in accordance with the present invention comprises at least 18 Tal effector motifs. In other words, the DNA-binding domain of a Tal effector protein within said fusion (poly)peptide is comprised of at least 18 Tal effector motifs. In the case of fusion (poly)peptides consisting of dimers as described above this means that each fusion (poly)peptide monomer comprises at least nine Tal effector motifs. More preferably, each fusion (poly)peptide comprises at least 12 Tal effector motifs, such as for example at least 14 or at least 16 Tal effector motifs. Methods for testing the DNA-binding specificity of a fusion (poly)peptide in accordance with the present invention are known to the skilled person and include, without being limiting, transcriptional reporter gene assays and electrophoretic mobility shift assays (EMSA).

Preferably, the binding site of the fusion (poly)peptide is up to 500 nucleotides, such as up to 250 nucleotides, up to 100 nucleotides, up to 50 nucleotides, up to 25 nucleotides, up to 10 nucleotides such as up to 5 nucleotides upstream (i.e. 5′) or downstream (i.e. 3′) of the nucleotide(s) that is/are modified in accordance with the present invention.

The above is mutatis mutandis also applicable with regard to the zinc-finger nuclease to be used in the method of the invention.

The term “zinc-finger nucleases” is well-known in the art and refers to a fusion (poly)peptide comprising a DNA-binding domain, wherein the DNA-binding domain comprises or consists of zinc finger repeats and the non-specific cleavage domain of a restriction nuclease. The zinc finger fusion (poly)peptide employed in the method of the invention retains or essentially retains the enzymatic activity of the native (restriction) endonuclease. In accordance with the present invention, (restriction) endonuclease function is essentially retained if at least 60% of the biological activity of the endonuclease activity are retained. Preferably, at least 75% or at least 80% of the endonuclease activity are retained. More preferred is that at least 90% such as at least 95%, even more preferred at least 98% such as at least 99% of the biological activity of the endonuclease are retained. Most preferred is that the biological activity is fully, i.e. to 100%, retained. Also in accordance with the invention, fusion (poly)peptides having an increased biological activity compared to the endogenous endonuclease, i.e. more than 100% activity. Methods of assessing biological activity of (restriction) endonucleases are well known to the person skilled in the art and include, without being limiting, the incubation of an endonuclease with recombinant DNA and the analysis of the reaction products by gel electrophoresis (Bloch K D.; Curr Protoc Mol Biol 2001; Chapter 3:Unit 3.2).

Without wishing to be bound by theory, the present inventors believe that the mechanism of double-strand cleavage by a TAL or zinc-finger fusion (poly)peptide requires dimerisation of the nuclease domain in order to cut the DNA substrate. Thus, in a preferred embodiment, at least two fusion (poly)peptides are introduced into the cell in step (a). Dimerisation of the fusion (poly)peptide can result in the formation of homodimers if only one type of fusion (poly)peptide is present or in the formation of heterodimers, when different types of fusion (poly)peptides are present. It is preferred in accordance with the present invention that at least two different types of fusion (poly)peptides having differing DNA-binding domains of a Tal effector protein or zinc-finger repeats are introduced into the cell. The at least two different types of fusion (poly)peptides can be introduced into the cell either separately or together. Also envisaged herein is a fusion (poly)peptide, which is provided as a functional dimer via linkage of two subunits of identical or different fusion (poly)peptides prior to introduction into the cell. Suitable linkers have been defined above.

The term “nucleic acid molecules encoding said TAL nucleases, zinc-finger nucleases or engineered meganucleases in expressible form” refers to a nucleic acid molecule which, upon expression in a cell or a cell-free system, results in a functional TAL nuclease, zinc-finger nuclease or engineered meganuclease. Nucleic acid molecules as well as nucleic acid sequences, as used throughout the present description, include DNA, such as cDNA or genomic DNA, and RNA. Preferably, embodiments reciting “RNA” are directed to mRNA. Furthermore included is genomic RNA, such as in case of RNA of RNA viruses.

It will be readily appreciated by the skilled person that more than one nucleic acid molecule may encode a fusion (poly)peptide due to the degeneracy of the genetic code. Degeneracy results because a triplet code designates 20 amino acids and a stop codon. Because four bases exist which are utilized to encode genetic information, triplet codons are required to produce at least 21 different codes. The possible 4³ possibilities for bases in triplets give 64 possible codons, meaning that some degeneracy must exist. As a result, some amino acids are encoded by more than one triplet, i.e. by up to six. The degeneracy mostly arises from alterations in the third position in a triplet. This means that nucleic acid molecules having different sequences, but still encoding the same fusion (poly)peptide can be employed in accordance with the present invention.

The term “engineered meganucleases” is well known in the art and used in accordance with said meaning herein. Briefly, it refers to meganucleases (endodeoxyribonucleases) that are characterized by a large DNA recognition site specifically recognizing between 12 to 40 base pairs of DNA. Because of the large recognition site, the sequence recognized by the meganucleases are unique in the genomes of many genera including mammals. On this basis, meganucleases (in particular of the LAGLIDADG family of homing endonucleases) have become a useful tool in specific gene targeting and efforts to change the specificity of the DNA recognition have been successfully undertaken. The best characterized endonucleases that are used in research and genome engineering include, e.g., I-SceI (from Saccharomyces cerevisiae), I-CreI (from Chlamydomonas reinhardtii) and I-DmoI (from Desulfurococcus mobilis). However, the meganuclease-induced genetic recombinations that can be performed are limited by the repertoire of meganucleases available. By modifying their recognition sequence through protein engineering, the targeted sequence can be changed to the desired recognition sites. The most advanced research and applications concern homing endonucleases from the LAGLIDADG family. To create tailor-made meganucleases, two approaches are currently used: 1. Modifying the specificity of existing meganucleases by introducing a number of variations to the amino acid sequence and then selecting the functional proteins on variations of the natural recognition site, 2. associating or fusing protein domains from different enzymes. This shuffling enables to develop chimeric meganucleases with a new recognition site composed of a half-site of meganuclease 1 and a half-site of protein 2. By fusing the protein domains of 1-DmoI and I-CreI, two chimeric meganucleases have been created using this method: E-DreI and DmoCre.

Preferred in the method of the invention is the use of fusion (poly)peptides with e.g., Tal effector motifs of a TAL effector protein, zinc-finger repeats, the helix-turn-helix (HTH) motif of homeodomains or the ribbon-helix-helix (RHH) motif as DNA-binding domain, and as nuclease domain a (poly)peptide that is encoded by a nucleic acid molecule encoding (I) a (poly)peptide having the activity of an endonuclease, which is (a) a nucleic acid molecule encoding a (poly)peptide comprising or consisting of the amino acid sequence of SEQ ID NO: 5; (b) a nucleic acid molecule comprising or consisting of the nucleotide sequence of SEQ ID NO: 6; (c) a nucleic acid molecule encoding an endonuclease, the amino acid sequence of which is at least 70% identical to the amino acid sequence of SEQ ID NO: 5; (d) a nucleic acid molecule comprising or consisting of a nucleotide sequence which is at least 50% identical to the nucleotide sequence of SEQ ID NO: 6; (e) a nucleic acid molecule which is degenerate with respect to the nucleic acid molecule of (d); or (f) a nucleic acid molecule corresponding to the nucleic acid molecule of any one of (a) to (e) wherein T is replaced by U; (II) a fragment of the (poly)peptide of (I) having the activity of an endonuclease. Said (poly)peptide represents the nuclease domain of a novel nuclease termed “Clo051” having the amino acid sequence of SEQ ID NO: 7. For example, a (poly)peptide having the activity of an endonuclease can be a (poly)peptide having or comprising the sequence of SEQ ID NO:7.

As defined herein above, certain amino acid sequence identities are envisaged in association with the Clo051 nuclease domain. Also envisaged in this regard are—with increasing preference—amino acid sequence identities of at least 75%, at least 80%, at least 85%, at least 90%, at least 95%, at least 97.5%, at least 98%, at least 98.5%, at least 99%, at least 99.5%, at least 99.8%, and 100% identity to the respective amino acid sequence.

As defined in the embodiments herein above, certain nucleotide sequence identities are envisaged in association with the Clo051 nuclease domain. Also envisaged in this regard are—with increasing preference—nucleotide sequence identities of at least 55%, at least 60%, at least 65%, at least 70%, at least 75%, at least 80%, at least 85%, at least 90%, at least 95%, at least 97.5%, at least 98%, at least 98.5%, at least 99%, at least 99.5%, at least 99.8%, and 100% identity to the respective nucleic acid sequence in accordance with the invention.

Fragments according to this aspect of the present invention are (poly)peptides having the activity of an endonuclease as defined herein above and comprise at least 90 amino acids. In this regard, it is preferred—with increasing preference—that the fragments according the present invention are (poly)peptides of at least 100, at least 125, at least 150, at least 200 amino acids, at least 300 amino acids, at least 400 amino acids. Fragments of said (poly)peptide, which substantially retain endonuclease activity, include N-terminal truncations, C-terminal truncations, amino acid substitutions, internal deletions and addition of amino acids (either internally or at either terminus of the protein). For example, conservative amino acid substitutions are known in the art and may be introduced into the endonuclease of the invention without substantially affecting endonuclease activity, i.e. reducing said activity.

Preferably, and with regard to item (I)(c) of said amino acid sequence having at least 70% sequence identity to SEQ ID NO: 5, the amino acid residues P66, D67, D84 and/or K86 of SEQ ID NO: 5 are not modified.

The use of the above-mentioned fusion (poly)peptides or engineered meganucleases in the method of the invention is preferred, because they are established genetic engineering tools and because of their specificity that may be changed depending on the target sequence that is the subject of modification according to the method of the invention.

In a more preferred embodiment of the method of the invention, the zinc-finger nucleases or TAL nucleases are fusion (poly)peptides of target sequence specific zinc-finger or TAL DNA binding domains and (a) a (poly)peptide comprising or consisting of the cleavage domain of the FokI endonuclease; or (b) a (poly)peptide that is encoded by a nucleic acid molecule encoding (I) a (poly)peptide having the activity of an endonuclease, which is (a) a nucleic acid molecule encoding a (poly)peptide comprising or consisting of the amino acid sequence of SEQ ID NO: 5; (b) a nucleic acid molecule comprising or consisting of the nucleotide sequence of SEQ ID NO: 6; (c) a nucleic acid molecule encoding an endonuclease, the amino acid sequence of which is at least 70% identical to the amino acid sequence of SEQ ID NO: 5; (d) a nucleic acid molecule comprising or consisting of a nucleotide sequence which is at least 50% identical to the nucleotide sequence of SEQ ID NO: 6; (e) a nucleic acid molecule which is degenerate with respect to the nucleic acid molecule of (d); or (f) a nucleic acid molecule corresponding to the nucleic acid molecule of any one of (a) to (e) wherein T is replaced by U; (II) a fragment of the (poly)peptide of (I) having the activity of an endonuclease.

FokI is a bacterial type IIS restriction endonuclease. It recognises the non-palindromic penta-deoxyribonucleotide 5′-GGATG-3′: 5′-CATCC-3′ in duplex DNA and cleaves 9/13 nucleotides downstream of the recognition site. FokI does not recognise any specific-sequence at the site of cleavage. The method of action and reaction conditions as well as reaction efficiencies have been well studied and established for various cell types. FokI is currently the only known typellS enzyme that has been determined to possess an isolated nuclease domain that works as a dimer (Bitinaite J. et al., Proc Natl Acad Sci USA 95:10570-10575 (1998); Doyon Y. et al. Nat Methods 8:74-79; Miller J C. Et al. Nat Biotechnol 25:778-785 (2007); Wah D A. Et al., Proc Natl Acad Sci USA 95:10564-10569 (1998)).

In a further preferred embodiment of the method of the invention, the activity of said NHEJ DNA repair complex in c. is decreased by decreasing the activity of NHEJ DNA ligase IV (LIG4).

The term “NHEJ DNA ligase IV” has been defined above in the context of the NHEJ DNA repair complex. LIG4 can either be directly targeted by one or more compounds that decrease its activity or said one or more compounds may target one of the interaction partners that are essential for and/or contribute to LIG4's capability to ligate the DNA strands resulting from a double-strand break, defined as “activity” of LIG4 herein, in the context of the NHEJ DNA repair complex. For example, indirectly decreasing the activity of LIG4 could involve, e.g., decreasing or inhibiting the expression of XRCC4 or the capability of XRCC4 to bind to LIG4. Directly decreasing the activity of LIG4 would involve, e.g., decreasing or inhibiting the expression of LIG4 or the capability of LIG4 to bind to cofactors essential and/or contributing to LIG4's activity, such as, e.g., XRCC4. The aggregate of LIG4 and cofactors that are essential and/or contribute to LIG4's activity is termed “functional LIG4 complex” in accordance with the invention. Targeting a cofactor that is essential for the activity of a LIG4 complex will abolish the activity of the LIG4 complex; targeting a cofactor contributing to a LIG4 complex will decrease the activity of said complex and thus the activity of the NHEJ DNA repair complex to the varying extents defined above.

In a more preferred embodiment of the method of the invention, the one or more compounds that decrease the activity of the non-homologous end joining (NHEJ) DNA repair complex are selected from the group consisting of small molecules, RNAi-molecules, antisense nucleic acid molecules, ribozymes, compounds inhibiting the formation of a functional LIG4 complex and/or compounds enhancing proteolytic degradation of LIG4.

A “small molecule” according to the present invention may be, for example, an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as less than about 500 amu, and even more preferably less than about 250 amu. The size of a small molecule can be determined by methods well-known in the art, e.g., mass spectrometry. The small molecules may be designed, for example, based on the crystal structure of the target molecule, where sites presumably responsible for the biological activity, can be identified and verified in in vivo assays such as in vivo high-throughput screening (HTS) assays.

In accordance with the present invention, the term “RNAi-molecules” refers to siRNA, shRNA or miRNA molecules. The term “siRNA (small interfering RNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene of which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1-5 nucleotides, more preferably from 1-3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art and depends on the envisioned application of the method of the invention. As described herein-above, there are several methods to introduce nuclei acid sequences into cells, including uptake of naked nuclei acids by endogenous cellular mechanisms. Taking advantage of said mechanisms, one may introduce RNAi-molecules into mammals, for example, by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules which, as endogenous RNA molecules, regulate gene expression. Binding to a complementary mRNA transcript triggers the degradation of said mRNA transcript through a process similar to RNA interference. Accordingly, miRNA may be employed to decrease the activity of NHEJ DNA ligase IV.

The term “antisense nucleic acid molecule” is known in the art and refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

A ribozyme (from ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in the last 10 years. The hammerhead ribozymes are characterized best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: An interesting region of the RNA, which contains the GUC (or CUC) triplet, is selected. Two oligonucleotide strands, each usually with 6 to 8 nucleotides, are taken and the catalytic hammerhead sequence is inserted between them. Molecules of this type were synthesized for numerous target sequences. They showed catalytic activity in vitro and in some cases also in vivo. The best results are usually obtained with short ribozymes and target sequences.

A recent development, also useful in accordance with the present invention, is the combination of an aptamer recognizing a small compound with a hammerhead ribozyme. The conformational change induced in the aptamer upon binding the target molecule is supposed to regulate the catalytic function of the ribozyme.

In accordance with the invention, modified versions of the above described RNAi molecules, antisense nucleic acid molecules and ribozymes are also fall under the definitions of the latter molecules and ribozymes given above. The term “modified versions” in accordance with the present invention refers to versions of said molecules that are modified to achieve i) modified spectrum of activity, organ specificity, and/or ii) improved potency, and/or iii) decreased toxicity (improved therapeutic index), and/or iv) decreased side effects, and/or v) modified onset of therapeutic action, duration of effect, and/or vi) modified pharmacokinetic parameters (resorption, distribution, metabolism and excretion), and/or vii) modified physico-chemical parameters (solubility, hygroscopicity, color, taste, odor, stability, state), and/or viii) improved general specificity, organ/tissue specificity, and/or ix) optimised application form and route by (a) esterification of carboxyl groups, or (b) esterification of hydroxyl groups with carboxylic acids, or (c) esterification of hydroxyl groups to, e.g. phosphates, pyrophosphates or sulfates or hemi-succinates, or (d) formation of pharmaceutically acceptable salts, or (e) formation of pharmaceutically acceptable complexes, or (f) synthesis of pharmacologically active polymers, or (g) introduction of hydrophilic moieties, or (h) introduction/exchange of substituents on aromates or side chains, change of substituent pattern, or (i) modification by introduction of isosteric or bioisosteric moieties, or (j) synthesis of homologous compounds, or (k) introduction of branched side chains, or (k) conversion of alkyl substituents to cyclic analogues, or (l) derivatisation of hydroxyl groups to ketales, acetales, or (m) N-acetylation to amides, phenylcarbamates, or (n) synthesis of Mannich bases, imines, or (O) transformation of ketones or aldehydes to Schiff's bases, oximes, acetales, ketales, enolesters, oxazolidines, thiazolidines; or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The term “compounds inhibiting the formation of a functional LIG4 complex” as used herein refers to compounds that either decrease or inhibit the expression of any interaction partners contributing to and/or making up said LIG4 complex as defined above or inhibit the interaction of said interaction partners with each other so as to prevent the formation of a functional LIG4 complex resulting in a decreased or abolished activity. Accordingly, RNAi-molecules, antisense molecules and/or ribozymes as described above can be used to decrease or inhibit expression of one or more interactions partners of the LIG4 complex. However, and with regard to inhibiting the binding of interaction partners to each other or inhibiting their biological activity, can be such as, for example, (poly)peptides resembling binding sites of interaction partners, antibodies, aptamers.

The term “antibody” as used in accordance with the present invention comprises, for example, polyclonal or monoclonal antibodies. Furthermore, also derivatives or fragments thereof, which still retain the binding specificity, are comprised in the term “antibody”. Antibody fragments or derivatives comprise, inter alia, Fab or Fab′ fragments as well as Fd, F(ab′)₂, Fv or scFv fragments; see, for example Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also includes embodiments such as chimeric (human constant domain, non-human variable domain), single chain and humanized (human antibody with the exception of non-human CDRs) antibodies.

Various techniques for the production of antibodies are well known in the art and described, e.g. in Harlow and Lane (1988) and (1999), loc. cit. Thus, the antibodies can be produced by peptidomimetics. Further, techniques described for the production of single chain antibodies (see, inter alia, U.S. Pat. No. 4,946,778) can be adapted to produce single chain antibodies specific for the target of this invention. Also, transgenic animals or plants (see, e.g., U.S. Pat. No. 6,080,560) may be used to express (humanized) antibodies specific for the target of this invention. Most preferably, the antibody is a monoclonal antibody, such as a human or humanized antibody. For the preparation of monoclonal antibodies, any technique which provides antibodies produced by continuous cell line cultures can be used. Examples for such techniques are described, e.g. in Harlow and Lane (1988) and (1999), loc. cit. and include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Surface plasmon resonance as employed in the BIAcore system can be used to increase the efficiency of phage antibodies which bind to an epitope of STIM2 or an epitope of a STIM2-regulated plasma membrane calcium channel (Schier, Human Antibodies Hybridomas 7 (1996), 97-105; Malmborg, J. Immunol. Methods 183 (1995), 7-13). It is also envisaged in the context of this invention that the term “antibody” comprises antibody constructs which may be expressed in cells, e.g. antibody constructs which may be transfected and/or transduced via, inter alia, viruses or plasmid vectors.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

More specifically, aptamers can be classified as nucleic acid aptamers, such as DNA or RNA aptamers, or peptide aptamers. Whereas the former normally consist of (usually short) strands of oligonucleotides, the latter preferably consist of a short variable peptide domain, attached at both ends to a protein scaffold.

Nucleic acid aptamers are nucleic acid species that, as a rule, have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers usually are peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys- loop in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

The term “peptide” as used herein describes a group of molecules consisting of up to 30 amino acids, whereas the term “protein” or “(poly)peptide” as used herein describes a group of molecules consisting of more than 30 amino acids. Peptides and proteins may further form dimers, trimers and higher oligomers, i.e. consisting of more than one molecule which may be identical or non-identical. The corresponding higher order structures are, consequently, termed homo- or heterodimers, homo- or heterotrimers etc. The terms “peptide” and “protein” (wherein “protein” is interchangeably used with “(poly)peptide” as defined above) also refer to naturally modified peptides/proteins wherein the modification is effected e.g. by glycosylation, acetylation, phosphorylation and the like. Such modifications are well-known in the art.

The term “compounds enhancing proteolytic degradation of a functional LIG4 complex” refers to compounds that modify LIG4 and/or interaction partners forming said LIG4 complex so that they are recognized and processed by a cellular proteolytic process. For example, the adenovirus E4-34k and E1b-55k oncoproteins associate into a ubiquitin-ligase that targets the host cell DNA-Ligase-IV for proteasomal degradation and thereby inhibit NHEJ DNA repair (Baker A. et al., 2007. J Virol 81:7034-7040 (2007); Cheng C Y. et al., J Virol 85:765-775 (2011); Forrester N A. et al., J Virol 85:2201-2211 (2011)).

In an even more preferred embodiment of the method of the invention, a small molecule is 6-Amino-2,3-dihydro-5-[(phenylmethylene)]amino]-2-4(1H)-pyrimidineone).

The small molecule defined by the above chemical designation has been shown to block the DNA binding of LIG4. The small molecule defined the above chemical designation has been identified by screening a library of chemical compounds and blocks a DNA-binding pocket within the DNA-binding domain of DNA ligase IV, acting as competitive inhibitor of DNA binding (Chen X. et al., 2008, Cancer Res 68:3169-3177).

In a further even more preferred embodiment of the method of the invention, the formation of a functional LIG4 complex can be inhibited by compounds that inhibit the binding of LIG4 to XRCC4 or inhibit the binding of Ku70 to Ku80.

As outlined above, one possibility to decrease the activity of the NHEJ DNA repair complex is to target the LIG4 complex within said NHEJ DNA repair complex by preventing interaction of the interaction partners of the LIG4 complex. XRCC4 and its interaction with LIG4 has been explained above evidencing the suitability to serve as target to decrease the activity of the NHEJ DNA repair complex. Potential compounds have also been described above and in more detail below.

Further preferred targets to decrease the activity of the NHEJ DNA repair complex are the interaction partners Ku70 and Ku80 in the LIG4 complex. For NHEJ-mediated repair, the Ku70/Ku80 (Ku) proteins bind with high affinity to DNA termini in a structure-specific manner and can promote end alignment of the two DNA ends. The DNA-bound Ku heterodimer recruits DNA-PKcs (DNA-dependent protein kinase catalytic subunit), and activates its kinase function. Together with the Artemis protein, DNA-PKcs can stimulate processing of the DNA ends. Finally, the XRCC4 (X-ray repair complementing defective repair in Chinese hamster cells 4)—DNA ligase IV complex, which does not form a stable complex with DNA but interacts stably with the Ku-DNA complex, carries out the ligation step to complete repair.

In a most preferred embodiment of the method of the invention, said compounds inhibiting the binding of LIG4 to XRCC4 or inhibiting the binding of Ku70 to Ku80 are (poly)peptides or nucleic acids encoding said (poly)peptides.

The (poly)peptides “inhibiting the binding of LIG4 to XRCC4 or inhibiting the binding of Ku70 to Ku80” are in accordance with the method of the invention preferably (poly)peptides such as those mentioned above, i.e. for example antibodies or aptamers.

In another most preferred embodiment, said (poly)peptides inhibiting the binding of (a) LIG4 to XRCC4 are the binding domains of LIG4 and/or XRCC4 mediating the binding of LIG4 to XRCC4; and/or (b) Ku70 to Ku80 are the binding domains of Ku70 and/or Ku80 mediating the binding of Ku70 to Ku80.

Peptides able to inhibit the binding of Lig4 to XRCC4 are, e.g., in mice peptides comprising or consisting of the residues 652-911 of the mouse DNA ligase IV protein (SEQ ID NO: 1), or subfragments thereof that can equally inhibit the binding of Lig4 to XRCC4, e.g. the 56 residue peptide comprising the residues 759-814 of the mouse DNA Ligase IV protein (SEQ ID NO: 2). The sequence starting from residue 652 to 911 of the mouse DNA ligase IV protein is as follows:

VNKVSNVFEDVEFCVMSGLDGYPKADLENRIAEFGGYIVQNPGPDTYC
VIAGSENVRVKNIISSDKNDVVKPEWLLECFKTKTCVPWQPRFMIHMC
PSTKQHFAREYDCYGDSYFVDTDLDQLKEVFLGIKPSEQQTPEEMAPV
IADLECRYSWDHSPLSMFRHYTIYLDLYAVINDLSSRIEATRLGITAL
ELRFHGAKVVSCLSEGVSHVIIGEDQRRVTDFKIFRRMLKKKFKILQES
WVSDSVDKGELQEENQYLL.

The 56 residue subfragment thereof as mentioned above has the sequence:

DCYGDSYFVDTDLDQLKEVFLGIKPSEQQTPEEMAPVIADLECRYSWDH
SPLSMFR.

Peptides able to inhibit the binding of Ku70 to Ku80 are, e.g., in mice peptides comprising or consisting of the residues 62-609 of mouse Ku70 (SEQ ID NO: 3) or subfragments thereof with maintained capability to bind to Ku80 at the site the peptide of SEQ ID NO: 3 binds. The sequence starting from residue 62 to 609 of the mouse Ku70 protein is as follows:

IQCIQSVYTSKIISSDRDLLAVVFYGTEKDKNSVNFKNIYVLQDLDN
PGAKRVLELDQFKGQQGKKHFRDTVGHGSDYSLSEVLWVCANLFS
DVQLKMSHKRIMLFTNEDDPHGRDSAKASRARTKASDLRDTGIFLD
LMHLKKPGGFDVSVFYRDIITTAEDEDLGVHFEESSKLEDLLRKVR
AKETKKRVLSRLKFKLGEDVVLMVGIYNLVQKANKPFPVRLYRETN
EPVKTKTRTFNVNTGSLLLPSDTKRSLTYGTRQIVLEKEETEELKR
FDEPGLILMGFKPTVMLKKQHYLRPSLFVYPEESLVSGSSTLFSALL
TKCVEKKVIAVCRYTPRKNVSPYFVALVPQEEELDDQNIQVTPGGFQ
LVFLPYADDKRKVPFTEKVTANQEQIDKMKAIVQKLRFTYRSDSFEN
PVLQQHFRNLEALALDMMESEQVVDLTLPKVEAIKKRLGSLADEFKE
LVYPPGYNPEGKVAKRKQDDEGSTSKKPKVELSEEELKAHFRKGTLG
KLTVPTLKDICKAHGLKSGPKKQELLDALIRHLEKN.

Peptides able to inhibit the binding of Ku80 to Ku70 are, e.g., in mice peptides comprising or consisting of residues 427 to 732 of mouse Ku80 (SEQ ID NO: 4) or subfragments thereof with maintained capability to bind to Ku70 at the site the peptide of SEQ ID NO: 4 binds. The sequence starting from residue 427 to 732 of the mouse Ku70 protein is as follows:

MEDLRQYMFSSLKNNKKCTPTEAQLSAIDDLIDSMSLVKKNEEEDIVE
DLFPTSKIPNPEFQRLYQCLLHRALHLQERLPPIQQHILNMLDPPTEMK
AKCESPLSKVKTLFPLTEVIKKKNQVTAQDVFQDNHEEGPAAKKYKTEK
EEDHISISSLAEGNITKVGSVNPVENFRFLVRQKIASFEEASLQLISHI
EQFLDTNETLYFMKSMDCIKAFREEAIQFSEEQRFNSFLEALREKVEIKQ
LNHFWEIVVQDGVTLITKDEGPGSSITAEEATKFLAPKDKAKEDTTGPEE
AGDVDDLLDMI.

The person skilled in the art is in the position to identify the homologous peptides in other species based on sequence alignment and sequence identity according to methods well-known in the art and described above.

Introducing corresponding (poly)peptides comprising or consisting of said binding areas results in competition of the natural ligands with the introduced (poly)peptides for the respective binding sites on the target molecule. Depending on the amount of competitive (poly)peptides introduced into a cell, the statistical occurrence of binding of a natural ligand or a competitive (poly)peptide can be influenced. For example, the more competitive (poly)peptides are introduced, the less natural ligands will bind to their natural target site. This way, one may also control the degree of the decrease of the activity of the NHEJ DNA repair complex if this is of importance.

Without being bound to or limited by a specific theory, this approach is considered to be both effective and harmless to the cell's physiology in comparison to, e.g., inhibiting expression of one of the interaction partners of the LIG4 complex.

In another most preferred embodiment of the method of the invention, said compounds enhancing proteolytic degradation of LIG4 are adenoviral (poly)peptides E1b55K and E4ORF6.

Adenoviral (poly)peptides E1b55K and E4ORF6 are known in the art and their mechanism of action has been described above.

Orthologues of E1b55K and E4ORF6 have been shown to exist in other adenoviral species with similar function (Cheng C Y. et al., J Virol 85:765-775 (2011); Forrester N A. et al., J Virol 85:2201-2211 (2011)). In accordance with the method of the invention, it is contemplated that—where possible—the cell used in the method of the invention is derived from mammal which represents a natural target for the adenovirus from which the adenoviral (poly)peptides are derived that are to be introduced in said cell. For example, if canine cells are to be used, then the adenoviral (poly)peptide E1b55K and/or E4ORF6 orthologues are derived from a canine adenovirus. However, also envisaged is the introduction of adenoviral (poly)peptides into a cell that is derived from a mammal that does not represent a natural target for the adenovirus from which said (poly)peptides are derived from. For example, human adenoviral (poly)peptides E1b55K and/or E4ORF6 may be introduced into murine cells.

In another most preferred embodiment of the method of the invention, said adenoviral (poly)peptides have been derived from a human adenovirus of serotype Ad9 or Ad16.

E1b55K and E4ORF6 (poly)peptides derived from Ad9 or Ad16 (17, 18) are considered to exclusively target LIG4 inhibiting its binding to XRCC4 thereby not showing any of the side effects that may be associated with using E1b55K and E4ORF6 (poly)peptides of other serotypes that are believed to also degrade the p53 protein. As will be appreciated by the skilled person, the adenoviral (poly)peptides E1b55K and/or E4ORF6 derived from a human adenovirus of serotypes Ad9 or Ad16 is a particularly valuable tool to decrease the activity of the NHEJ DNA repair complex when the method of the invention is performed in vivo, or alternatively ex vivo or in vitro with cells that are to be reintroduced into a mammal. For example, said mammals include humans, in particular human embryos or human oocytes.

In a preferred embodiment of the method of the invention, said mammalian cell is selected from the group consisting of an ungulate cell, a rodent cell, a rabbit cell, a primate cell or a human cell.

The skilled person is well-aware of what groups of mammals fall under the terms ungulates, rodents, rabbits and primates. Preferably, an ungulate cell is derived from horse, zebra, donkey, cattle, camel, goat, pig, sheep, giraffe, okapi, elk, deer, antelope or gazelle. Preferably, a primate cell is derived from rhesus macaques, green monkeys, chimpanzees, baboons, squirrel monkeys or marmosets as well as from lemurs, lorises, galagos or tarsiers. Preferably, a rodent cell is derived from a guinea pig, vole, porcupine, squirrel, chipmunk, beaver or mouse or rat (see below). Furthermore, mammalian cells may also be derived from cats.

In another preferred embodiment, the mammalian cell is a mouse or a rat cell, i.e. a specific rodent cell. The use of rats and mice as tools to investigate various aspects ranging from developmental studies to metabolic studies, drug safety and diseases. As a consequence, the method of the invention will provide significantly impact the availability, reproducibility and economics of generating mice or rats whose genome is modified. Because the genome of oocytes can be modified (as described below) genetically modified mice can be generated in a shorter time as compared to ES cell technology, the rate of modified mice per litter is greatly increased all of which affects the overall costs associated with the generation of a strain of mice having a modified genome.

In a preferred embodiment of the method of the invention, the mammalian cell is an embryonic stem cell or an oocyte.

The term “embryonic stem cell” is well known in the art and has been described herein above. As used herein the term “oocyte” refers to the female germ cell involved in reproduction, i.e. the ovum or egg cell. In accordance with the present invention, the term “oocyte” comprises both oocytes before fertilisation as well as fertilised oocytes, which are also called zygotes. Thus, the oocyte before fertilisation comprises only maternal chromosomes, whereas an oocyte after fertilisation comprises both maternal and paternal chromosomes. After fertilisation, the oocyte remains in a double-haploid status for several hours, in mice for example for up to 18 hours after fertilisation. The term “zygote” is also well-known in the art and refers to the initial cell formed when an oocyte and a sperm cell are joined by means of sexual reproduction. The fertilization of the oocyte by the sperm triggers egg activation to complete the transformation to a zygote by signaling the completion of meiosis and the formation of pronuclei. At this stage the zygote represents a 1-cell embryo that contains a haploid paternal pronucleus derived from the sperm and a haploid maternal pronucleus derived from the oocyte. After migration of the two pronuclei together, their membranes break down, and the two genomes condense into chromosomes, thereby reconstituting a diploid organism. In mice this totipotent single cell stage lasts for ˜18 hours until the first mitotic division occurs. As totipotent single entities, mammalian zygotes can be regarded as a preferred substrate for genome engineering since the germ line of the entire animal is accessible within a single cell. The invention is not limited to using mammalian zygotes, but may of course also use oocytes. Preferably, the oocyte is a fertilized mammalian oocyte. Also preferred is that the compounds used in a. and c. of the method of the invention or the nucleic acid molecules encoding said compounds as well as the DNA molecules in b. are introduced into the mammalian oocyte by microinjection. Microinjection into the oocyte can be carried out by injection into the nucleus (before fertilisation), the pronucleus (after fertilisation) and/or by injection into the cytoplasm (both before and after fertilisation). When a fertilised oocyte is employed, injection into the pronucleus is carried out either for one pronucleus or for both pronuclei. Injection of the compounds in a. of the method of modifying a target sequence of the present invention is preferably into the nucleus/pronucleus, while injection of an mRNA encoding said compounds of a. is preferably into the cytoplasm. Injection of the DNA molecule of b. is preferably into the nucleus/pronucleus. However, injection of the DNA molecule in b. can also be carried out into the cytoplasm when said DNA molecule is provided as a nucleic acid sequence having a nuclear localisation signal to ensure delivery into the nucleus/pronucleus. Preferably, the microinjection is carried out by injection into both the nucleus/pronucleus and the cytoplasm. For example, the needle can be introduced into the nucleus/pronucleus and a first amount of the compounds of a. and/or c. and/or the DNA molecule of b. are injected into the nucleus/pronucleus. While removing the needle from the oocyte, a second amount of the compounds of a. and/or c. and/or the DNA molecule of b. is injected into the cytoplasm. Methods for carrying out microinjection are well known in the art and are described for example in Nagy et al. (Nagy A, Gertsenstein M, Vintersten K, Behringer R., 2003. Manipulating the Mouse Embryo. Cold Spring Harbour, New York: Cold Spring Harbour Laboratory Press).

In further embodiment, the invention relates to a method of producing a non-human mammal carrying a modified target sequence in its genome, the method comprising transferring a cell produced by the method of the invention into a pseudo pregnant female host.

In accordance with the present invention, the term “transferring a cell produced by the method of the invention into a pseudopregnant female host” includes the transfer of a fertilised oocyte but also the transfer of pre-implantation embryos of for example the 2-cell, 4-cell, 8-cell, 16-cell and blastocyst (70- to 100-cell) stage. Said pre-implantation embryos can be obtained by culturing the cell under appropriate conditions for it to develop into a pre-implantation embryo. Furthermore, injection or fusion of the cell with a blastocyst are appropriate methods of obtaining a pre-implantation embryo. Where the cell produced by the method of the invention is a somatic cell, derivation of induced pluripotent stem cells is required prior to transferring the cell into a female host such as for example prior to culturing the cell or injection or fusion of the cell with a pre-implantation embryo. Methods for transferring an oocyte or pre-implantation embryo to a pseudo pregnant female host are well known in the art and are, for example, described in Nagy et al., (Nagy A, Gertsenstein M, Vintersten K, Behringer R., 2003. Manipulating the Mouse Embryo. Cold Spring Harbour, New York: Cold Spring Harbour Laboratory Press).

It is further envisaged in accordance with the method of producing a non-human mammal carrying a modified target sequence in its genome that a step of analysis of successful genomic modification is carried out before transplantation into the female host. As a non-limiting example, the oocyte can be cultured to the 2-cell, 4-cell or 8-cell stage and one cell can be removed without destroying or altering the resulting embryo. Analysis for the genomic constitution, e.g. the presence or absence of the genomic modification, can then be carried out using for example PCR or southern blotting techniques or any of the methods described herein above. Such methods of analysis of successful genotyping prior to transplantation are known in the art and are described, for example in Peippo et al. (Peippo J, Viitala S, Virta J, Raty M, Tammiranta N, Lamminen T, Aro J, Myllymaki H, Vilkki J.; Mol Reprod Dev 2007; 74:1373-1378).

Where the cell is an oocyte, the method of producing a non-human mammal carrying a modified target sequence in its genome comprises (a) modifying the target sequence in the genome of a mammalian oocyte in accordance with the method of the invention; (b) transferring the oocyte obtained in (a) to a pseudopregnant female host; and, optionally, (c) analysing the offspring delivered by the female host for the presence of the modification.

For this method of producing a non-human mammal, fertilisation of the oocyte is required. Said fertilisation can occur before the modification of the target sequence in step (a) in accordance with the method of producing a non-human mammal of the invention, i.e. a fertilised oocyte can be used for the method of modifying a target sequence in accordance with the invention. The fertilisation can also be carried out after the modification of the target sequence in step (a), i.e. a non-fertilised oocyte can be used for the method of modifying a target sequence in accordance with the invention, wherein the oocyte is subsequently fertilised before transfer into the pseudopregnant female host.

The step of analysing for the presence of the modification in the offspring delivered by the female host provides the necessary information whether or not the produced non-human mammal carries the modified target sequence in its genome. Thus, the presence of the modification is indicative of said offspring carrying a modified target sequence in its genome whereas the absence of the modification is indicative of said offspring not carrying the modified target sequence in its genome. Methods for analysing for the presence or absence of a modification have been detailed above.

The non-human mammal produced by the method of the invention is, inter alia, useful to study the function of genes of interest and the phenotypic expression/outcome of modifications of the genome in such animals. It is furthermore envisaged, that the non-human mammals of the invention can be employed as disease models and for testing therapeutic agents/compositions. Furthermore, the non-human mammal of the invention can also be used for livestock breeding.

Preferably, the method of producing a non-human mammal further comprises culturing the cell to form a pre-implantation embryo or introducing the cell into a blastocyst prior to transferring it into the pseudo pregnant female host. Methods for culturing the cell to form a pre-implantation embryo or introducing the cell into a blastocyst are well known in the art and are, for example, described in Nagy et al., loc. cit.

The term “introducing the cell into a blastocyst” as used herein encompasses injection of the cell into a blastocyst as well as fusion of a cell with a blastocyst. Methods of introducing a cell into a blastocyst are described in the art, for example in Nagy et al., loc. cit.

The present invention further relates to a non-human mammalian animal obtainable by the above described method of the invention.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show:

FIG. 1: Gene targeting in mammalian zygotes.

A: Upon the microinjection of zygotes with ZFN mRNA and a specific gene targeting vector double strand breaks (DSB) are introduced into the target gene. These breaks are mostly closed by the nonhomologous end joining repair pathway (NHEJ) prior to recombination with the gene targeting vector. Only a minor fraction of ZFN induced DSBs are repaired by the homologous recombination pathway (HR) using the targeting vector as repair template. Mice derived from the transfer of injected zygotes represent mostly non-targeted animals (wildtype or alleles with small deletions), with a fraction of −5% of targeted knockout (KO) or knockin (KI) mutants. B: Zygotes are coinjected with ZFN mRNA, gene targeting vector and molecules that lead to the inactivation of the NHEJ DNA repair pathway. NHEJ inhibition can be achieved by the coinjection of mRNA coding for inhibitory fragments of DNA ligase IV, XRCC4, Ku70 or Ku80, or synthetic peptides targeting the binding sites of DNA ligase IV to XRCC4 or of Ku70 to Ku80. Upon inhibition of NHEJ repair DSBs are resolved mostly by HR with the targeting vector, leading to a strong increase (30%) in the recovery of targeted mutants.

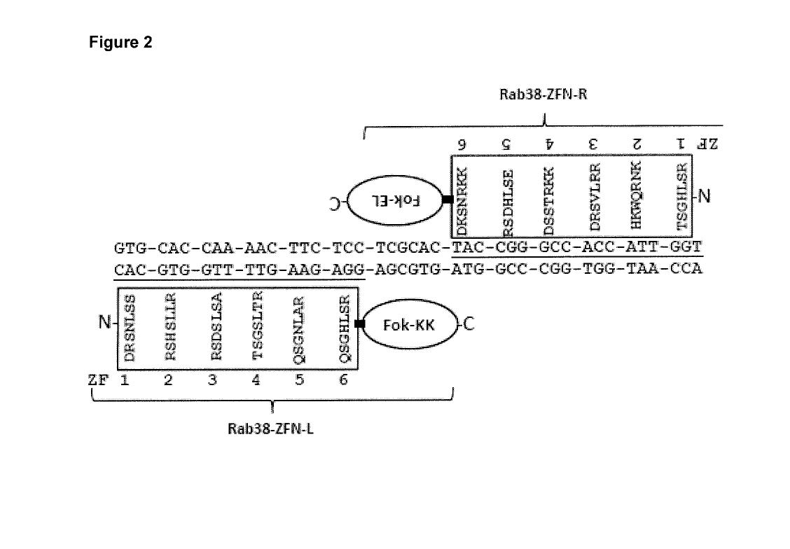

FIG. 2: Zinc-finger nucleases targeting the mouse Rab38 gene.

Zinc-finger nucleases recognizing a target sequence within exon 1 of the mouse Rab38 gene. Shown are six trinucleotide sequences (underlined) that are recognised by the indicated zinc-finger recognition helices of the ZFN-Rab38-L and -R fusion (poly)peptides. The zinc-finger domains of ZFN-Rab38-L and -R are C-terminally fused to the KK or EL double mutant nuclease domains of FokI nuclease. The two 18 bp target sequences are flanking a central 6 bp spacer sequence that is cut by the ZFN FokI domains.

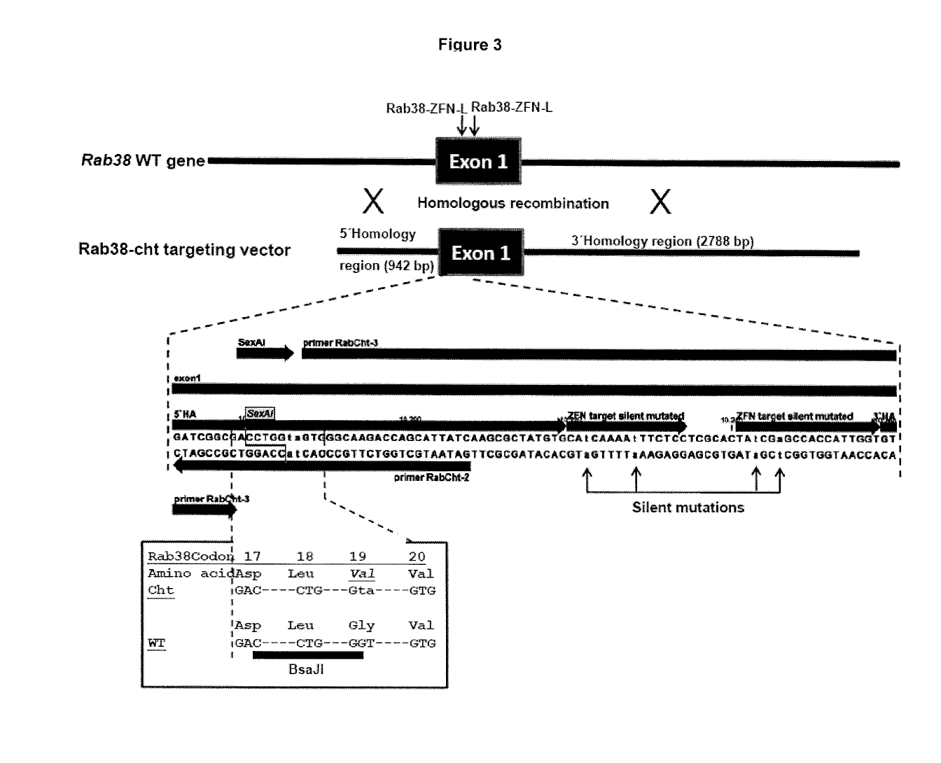

FIG. 3: Gene targeting vector for the mouse Rab38 gene.

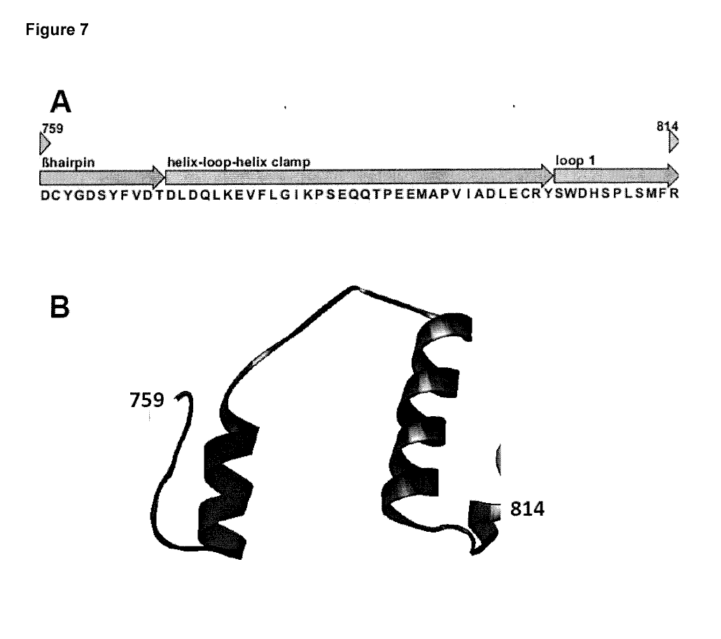

Within exon 1 of the wildtype Rab38 gene (Rab38 WT) the recognition sites for the zinc-finger nuclease pair Rab38-ZFN-L and -R (FIG. 7) are indicated. The Rab38-cht targeting vector contains a 942 bp 5′-homology region and a 2788 bp 3′-homology region flanking the Rab38-ZFN recognition sites. Within exon1 two nucleotide changes within codon 19 (Gta) of Rab38 create a chocolate (cht) missense mutation coding for valine (Val) instead of the wildtype (WT) glycine (Gly), and remove a BsaJI restriction site. In each of the adjacent Rab38-ZFN recognition sites two silent mutations are introduced to prevent the binding of Rab38-ZFN's to the targeting vector. In addition, the regions encompassing the PCR primers RabCht-2 and RabCht-3, which were used for vector contruction, are shown. The induction of a double-strand break within the wildtype Rab38 gene by the Rab38-ZFN pair stimulates homologous recombination of the Rab38-cht targeting vector and integrates the chocolate missense and the silent mutations into the genome.

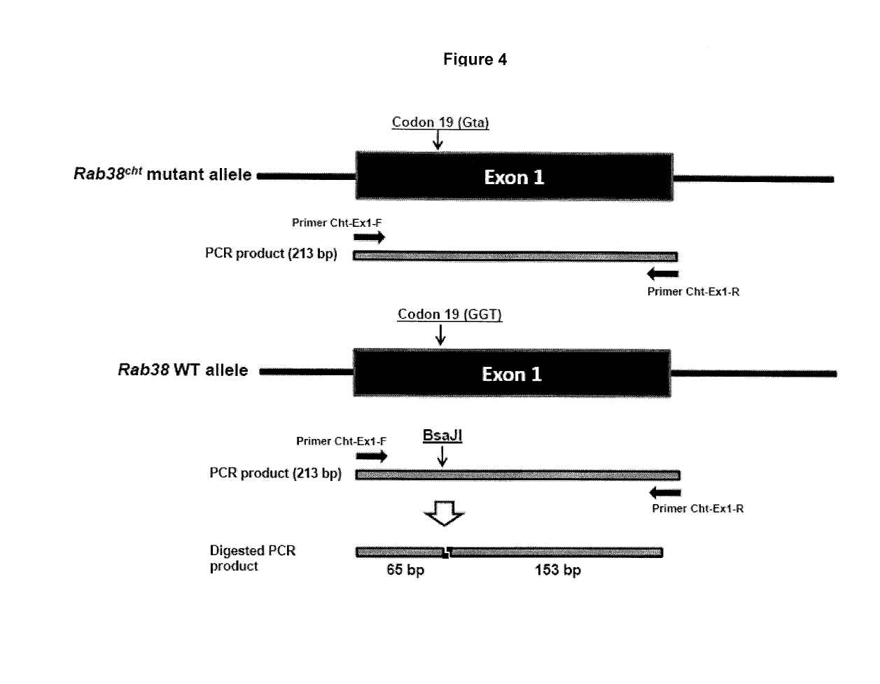

FIG. 4: Genotyping assay for the induced Rab38 chocolate mutation.

Using genomic DNA of mice derived from fertilised oocytes injected with the Rab38-cht gene targeting vector and mRNA for Rab-ZFN-L and -R, exon1 of Rab38 is amplified by PCR using the primer pair Cht-Ex1-F and Cht-Ex1-R. The 213 bp PCR is subsequently digested with BsaJI and analysed by gel electrophoresis. PCR fragments derived from the Rab38 wildtype allele that contains a BsaJI site are cut into two subfragments of 65 and 153 bp. PCR products derived from the Rab38 chocolate allele can not be digested with BsaJI since its recognition site is lost by the nucleotide exchanges within codon 19.

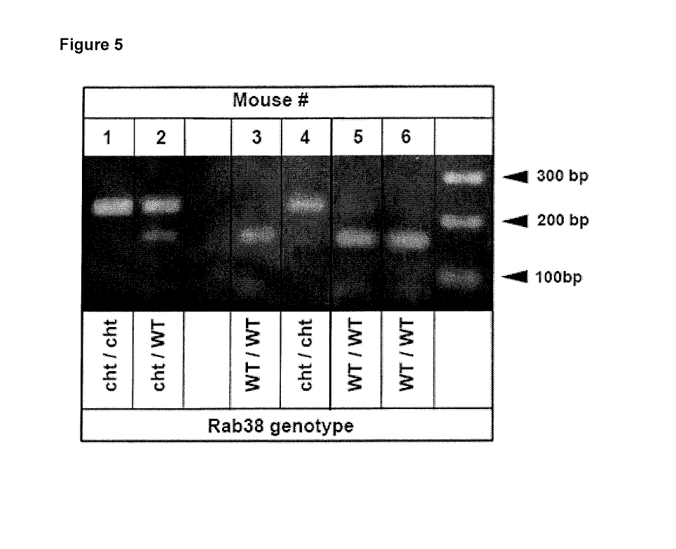

FIG. 5: Genotyping of Rab38 alleles in mice derived from microinjected fertilised oocytes.

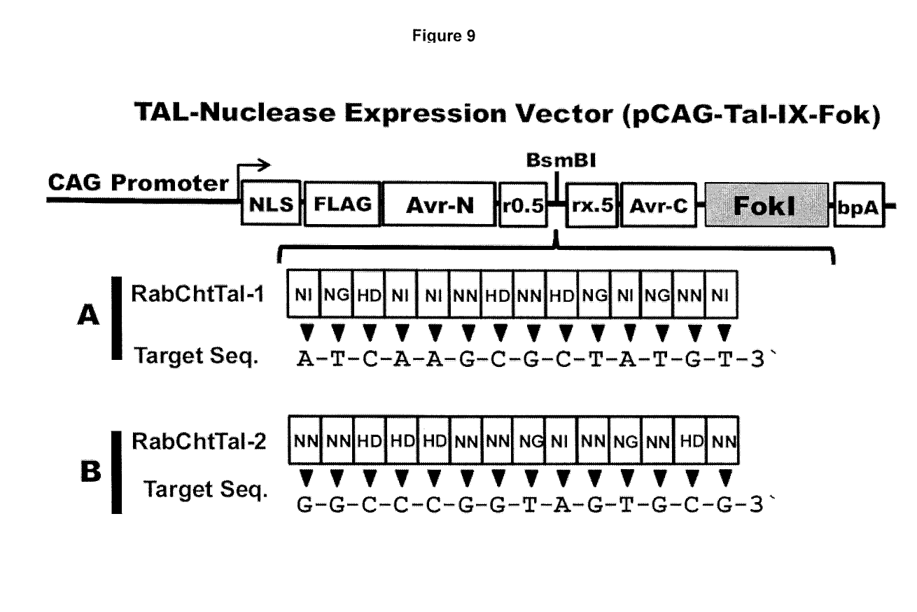

Fertilised oocytes were injected with Rab38-cht gene targeting vector and mRNAs coding for ZFN-Rab38-L and -R. Upon the transfer of injected embryos into pseuopregnant females live pups were obtained and their tail DNA was used to genotype for the presence of the Rab38 chocolate mutation following the approach shown in FIG. 9. In the presented results six mice were analysed, two of which exhibit a homozygous chocolate (cht/cht) or wildtype (WT/WT) genotype whereas one individual was heterozygous for the cht mutation (cht/WT).

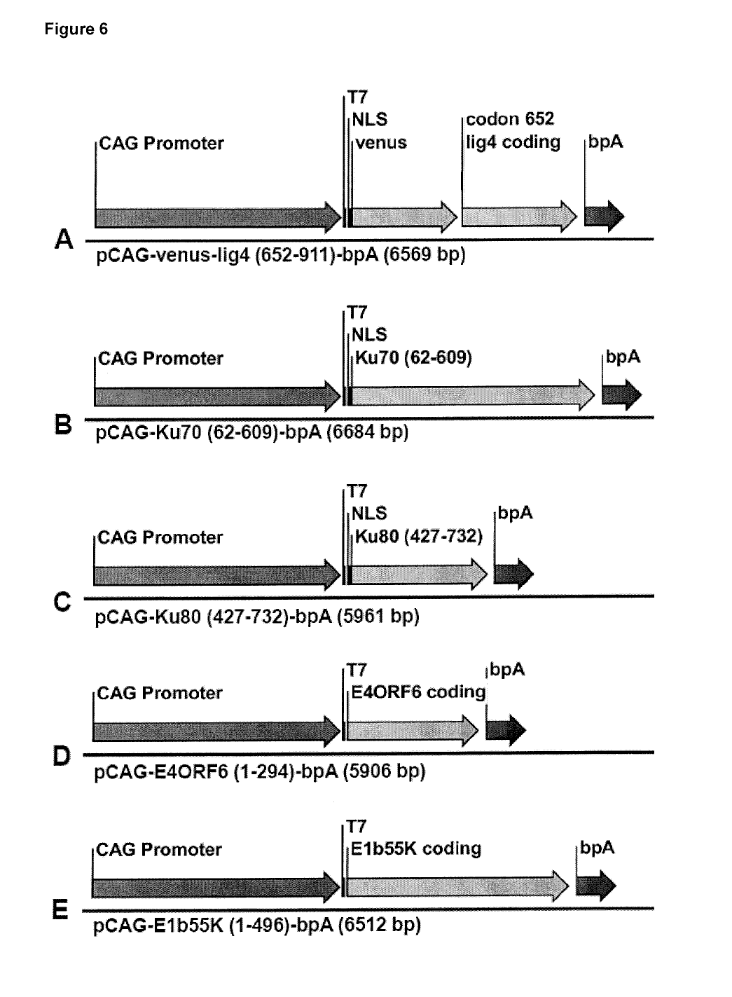

FIG. 6: Plasmids for the production of DNA ligase IV, Ku70, Ku80, E1b55K and E4ORF6 protein fragments.

The plasmids contain a CAG promoter region and a bovine polyadenylation signal (bpA), flanking the coding region, for protein expression in mammalian cells. A T7 polymerase promoter (T7) upstream of the ATG start codon allows the in vitro transcription of the coding regions as mRNA. Plasmid pCAG-venus-lig4-bpa (A) contains the coding sequence for the residues 652-911 of mouse DNA ligase IV, fused to a GFP (venus) reporter domain, pCAG-Ku70-bpA (B) contains the coding sequence for the residues 62-609 of mouse Ku70, pCAG-Ku80-bpA (C) contains the coding sequence for the residues 427-732 of mouse Ku80, pCAG-E4ORF6-bpA (D) contains the complete coding sequence (1-294) for the Adenovirus-5 E4ORF6 protein and pCAG-E1b55K-bpA (E) contains the complete coding sequence (1-496) for the Adenovirus-5 E1b (55K) protein. The coding regions of plasmids A-C are fused to a nuclear localisation peptide (NLS).

FIG. 7: Peptide for the disruption of the DNA ligase IV/XRCC4 protein complex.

A: The 56 residue peptide comprises the residues 759-814 of the mouse DNA Ligase IV protein. This peptide folds into a hairpin, helix-loop-helix and a loop structure (B), covering the binding site of DNA ligase IV to XRCC4.

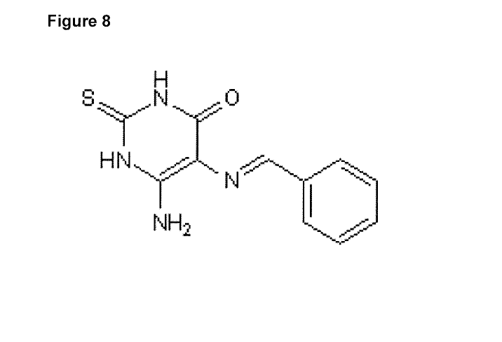

FIG. 8: Structure of the DNA ligase inhibitor L189.

Chemical structure of compound L189 (6-Amino-2,3-dihydro-5-[(phenylmethylene)amino]-2-4(1H)-pyrimidineone) (CAS No 64232-83-3) that blocks the catalytic center of DNA ligase IV.

FIG. 9: TAL-FokI nuclease expression vectors.

The Tal nuclease expression vector pCAG-Tal-IX-Fok contains a CAG promoter region and a transcriptional unit comprising, upstream of a central pair of BsmBI restriction sites, an ATG start codon (arrow), a nuclear localisation sequence (NLS), a FLAG Tag sequence (FLAG), a linker, a segment coding for 110 amino acids of the Tal protein AvrBs3 (AvrN) and its invariable N-terminal Tal repeat (r0.5). Downstream of the BsmBI sites the transcriptional unit contains an invariable C-terminal Tal repeat (rx.5), a segment coding for 44 amino acids derived from the Tal protein AvrBs3, the coding sequence of the FokI nuclease domain and a polyadenylation signal sequence (bpA). DNA segments coding for Tal repeats can be inserted into the BsmBI sites of pCAG-Tal-IX-Fok for the expression of variable Tal-Fok nuclease fusion proteins. A: to create the RabChtTal1 nuclease an array of 14 Tal repeats recognising the indicated target sequence was inserted into pCAG-Tal-IX-Fok. B: to create the RabChtTal-2 nuclease an array of 14 Tal repeats recognising the indicated target sequence was inserted into pCAG-Tal-IX-Fok. Each 34 amino acid Tal repeat is drawn as a square indicating the repeat's amino acid code at positions 12/13 that confers binding to one of the DNA nucleotides of the target sequence (NI>A or NS>A, NG>T, HD>C, NN>G) shown below.

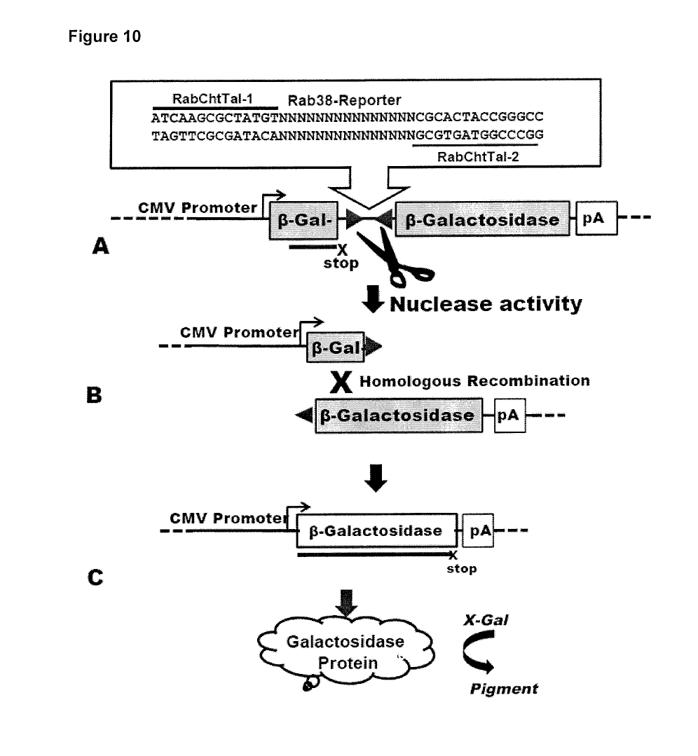

FIG. 10: Tal nuclease reporter assay.

A: The Tal nuclease reporter plasmid contains a CMV promoter region, a 400 bp sequence coding for the N-terminal segment of 3-galactosidase and a stop codon. This unit is followed by the Tal nuclease target region consisting of recognition sequences (underlined) for the RabChtTal1 and RabChtTal-2 nucleases that are separated by a 15 bp spacer region (NNN . . . ). The Tal nuclease target region is followed by the complete coding region for β-galactosidase and a polyadenylation signal (pA). To test for nuclease activity against the target sequence a pair of Tal nuclease expression vector (FIG. 9) is transiently transfected into HEK 293 cells that contain genomically integrated copies of the corresponding reporter plasmid. Upon expression of the Tal nuclease protein the reporter DNA is opened by a nuclease induced double strand-break within the Tal nuclease target sequence (scissor). B: The DNA regions adjacent to the double-strand break are identical over 400 bp and can be aligned and recombined (X) by homologous recombination DNA repair. C: Homologous recombination of an opened reporter construct results into a functional β-galactosidase expression vector that produces the β-galactosidase enzyme. After two days the transfected cell population is fixed and recombined cells can be visualized by histochemical (X-Gal) staining.

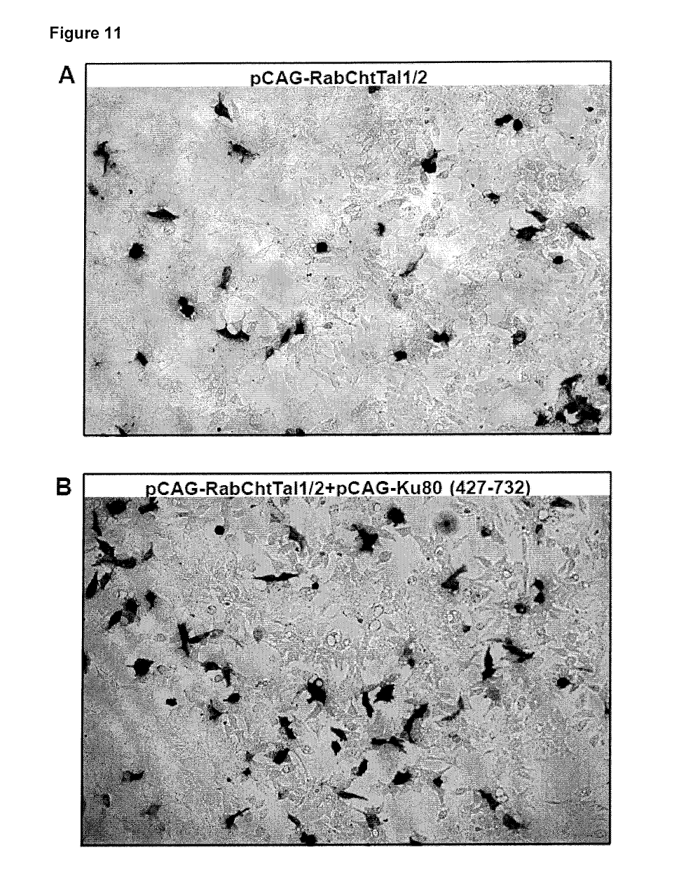

FIG. 11: Activity of Tal nucleases in HEK 293 cells.

To test for the effect of Ku80 inhibition, HEK293 cells harboring genomic integrated copies of the Rab reporter construct (FIG. 10) were transfected with expression vectors for the RabChtTal-1 and -2 nucleases without or together with the expression vector pCAG-Ku80 (427-732)-bpA (FIG. 6). Specific nuclease activity against the reporter's target sequence leads to homologous recombination and the expression of β-galactosidase. Two days after transfection the cell populations were fixed and the fraction of □β-galactosidase expressing cells was determined by histochemical X-Gal staining. A: X-Gal stained reporter cell culture upon transfection with RabChtTal-1 and -2 nuclease expression vectors. Cells were counted from two representative images indicating a recombination rate of 4.89% (139 positive cells of 2838 cells in total). B: X-Gal stained reporter cell culture upon transfection with RabChtTal-1 and -2 nuclease expression vectors together with pCAG-Ku80(427-732)-bpA. Cells were counted from two representative images indicating a recombination rate of 9.50% (270 positive cells of 2842 cells in total).

The examples illustrate the invention:

### EXAMPLE 1

A) Introduction of a Missense Mutation into the First Exon of the Mouse Rab38 Gene

To demonstrate the utility of the zinc-finger nuclease technique we selected the Rab38 gene, encoding the RAB38 protein that is a member of a family of proteins known to play a crucial role in vesicular trafficking. In chocolate (cht) mutant mice a single nucleotide exchange at position 146 (G>T mutation) within the first exon of Rab38 leads to the replacement of glycine by valine at codon 19 (19). This amino acid replacement is located within the conserved GTP binding domain of RAB38 and impairs the sorting of the tyrosinase-related protein 1 (TYRP1) into the melanosomes of Rab38^{cht}/Rab38^{cht} melanocytes. TYRP1 is a melanosomal membrane glycoprotein, which functions both as a 5,6-Dihydroxyindol-2-carbonic-acid oxidase enzyme to produce melanin and as a provider of structural stability to tyrosinase in the melanogenic enzyme complex. TYRP1 is believed to transit from the trans-Golgi network to stage II melanosomes by means of clathrin-coated vesicles. The reduced amount of correctly located TYRP1 leads to an impairment of pigment production and the change of fur color from black to a chocolate-like brown color in Rab38^{cht}/Rab38^{cht} mice. Since mutations of genes needed for melanocyte function are known to cause oculocutaneous albinism (OCD), such as Hermansky-Pudlak syndrome in man, the Rab38 gene is a candidate locus in OCD patients (19).

We aimed to introduce a phenocopy of the chocolate mutation at codon 19 of Rab38 using a pair of zinc-finger nucleases (ZFN-Rab38-L, -R) that each recognise via six zinc-finger domains a 18 bp target sequence located up- and downstream of the central 6 bp spacer sequence 5′-TCGCAC-3′ within exon 1 of Rab38 (FIG. 2) (6). Expression constructs for these zinc-finger nucleases were obtained by gene synthesis from a commercial service provider.

For the modification of Rab38 by homologous recombination in fertilised oocytes we constructed the gene targeting vector Rab38-cht (SEQ ID NO: 8), comprised of two homology regions encompassing 942 and 2788 bp of genomic sequence flanking exon1 of the mouse Rab38 gene (SEQ ID NO: 9). For this purpose the vectors 5′- and 3′-homology arms were amplified from the genomic BAC clone RPCI-421G2 (derived from the C57BL/6J genome, Imagenes GmbH, Berlin) using the primer pair RabCht-1 (SEQ ID NO: 10) & RabCht-2 (SEQ ID NO: 11), and the primer pair RabCht-3 (SEQ ID NO: 12) & RabCht-4 (SEQ ID NO: 13). Primers RabCht-2 and -3 were selected such that they overlap by 21 bp within exon1 of Rab38, immediately downstream of codon 19 (FIG. 3). Within the sequence of codon 19 primer 2 contained two nucleotide changes that modify codon 19 from the wildtype sequence GGT, coding for glycine, into GTA, coding for valine. This new chocolate mutation can be distinguished from the natural chocolate mutation, which exhibits only a single nucleotide exchange within codon 19 (GTT) coding for valine (19). Both chocolate mutant alleles can be further distinguished from the wildtype allele by restriction analysis since the mutations in codon 19 remove a recognition site for the restriction endonuclease BsaJI (FIG. 3). The recognition region for the ZFN-Rab38-L and -R zinc-finger proteins is located 33 bp downstream of codon 19 (FIG. 3). For the construction of the targeting vector 3′-homology region each 18 bp ZFN recognition sequence was further modified by the introduction of two silent nucleotide changes that do not alter the RAB38 protein sequence (FIG. 3), in order to avoid the potential processing of the targeting vector by the Rab38 specific ZFNs. To construct the complete Rab38-cht targeting vector the PCR products representing the 5′- and 3″-homology arms were fused by a fusion PCR method using primers RabCht-1 and -4 for amplification. Since primer 1 includes an I-SceI restriction site and primer 4 a SalI restriction site, it was possible to clone the I-SceI+SalI digested 3.7 kb PCR product into the backbone of the vector pRosa26.3-3 (13) that was opened with I-SceI and SalI. The integrity of the completed vector was confirmed by DNA sequencing.

B) Targeting of Rab38 in Zygotes without Inhibition of NHEJ DNA Repair Proteins

For microinjection into fertilised oocytes the circular Rab38-cht vector DNA (15 ng/μl) was mixed with in vitro transcribed mRNA coding for ZFN-Rab38-L and -R (each 3 ng/μl) in injection buffer as described (13). Upon microinjection the zincfinger nuclease mRNAs are translated into proteins that induce a double strand break at one or both Rab38 alleles in one or more cells of the developing embryo. This event stimulates the recombination of the Rab38-cht targeting vector with a Rab38 allele via the homology regions present in the vector and leads to the site-specific insertion of the mutant codon 19 into the genome, resulting into a Rab38^{cht} allele bearing the chocolate mutation (FIG. 3). The microinjected zygotes were transferred into pseudopregnant females to allow their further development into live mice. From the resulting mice genomic DNA was extracted from tail tips to analyse for the presence of the desired homologous recombination event at the Rab38 locus by PCR. This analysis was performed by the PCR amplification of the genomic region encompassing exons using the primer cht-Ex1F (SEQ ID NO: 14) and primer cht-Ex1R (SEQ ID NO: 15) (FIG. 4). From both alleles, the Rab38 wildtype gene and the Rab38^{cht} allele, the resulting PCR products have a length of 213 bp. However, the presence of a Rab38^{cht} allele can be recognised upon digestion of the PCR products with BsaJI, since the Rab38^{cht} mutation at codon 19 leads to the removal of a BsaJI restriction site that is present in the wildtype sequence. Therefore, PCR products amplified from the Rab38 wildtype allele can be digested with BsaJI into two subfragments of 65 bp and 148 bp whereas PCR products amplified from the Rab38^{cht} allele are resistant to BsaJI digestion (FIG. 4).

In one such experiment, 52 mice derived from microinjected zygotes were analysed by the Rab38 PCR assay. Among this group 49 mice exhibited two alleles of the normal Rab38 wildtype genotype, whereas 3 individuals harboured one allele of the preplanned Rab38 chocolate mutation, as indicated by the absence of the BsaJI restriction site in exon 1. An example of these genotyping results is shown in FIG. 5.

Taken together, it was possible to introduce a preplanned modification into the coding region of the Rab38 gene by zinc-finger nuclease assisted homologous recombination in fertilised oocytes. The frequency of targeted mutagenesis in the absence of NHEJ repair inhibition was in the range of 5% (3 mutants/49 wildtype mice).

C) Targeting of Rab38 in Zygotes with Inhibition of NHEJ DNA Repair Proteins

To improve the rate of homologus recombination of the Rab38-cht vector with the Rab38 gene in fertilised oocytes, these were microinjected with ZFN-Rab38-L and -R mRNA and targeting vector together with molecules inactivating DNA ligase IV or Ku70 or Ku80 activity to interfere with NHEJ DNA repair. DNA ligase IV acts in a multimeric complex with the XRCC4 protein and Ku70 interacts with Ku80 in a dimeric complex while their monomers are biologically inactive (20). The binding interface of DNA Ligase IV/XRCC4 and the Ku70/Ku80 proteins have been characterised (21). The overexpression of the binding domains of DNA ligase IV, Ku70 or Ku80 competes in a dominant negative manner with the binding of the full length proteins (21-23). Thereby the biological activities of the DNA ligase IV/XRCC4 or Ku70/Ku80 complexes and subsequently the efficacy of NHEJ DNA repair become suppressed.

To interfere with DNA ligase IV activity in the pronucleus of fertilised oocytes, we constructed plasmid pCAG-venus-lig4-bpA (FIG. 6A) (SEQ ID NO: 16) that contains the C-terminal part (residues 652-911) of the coding region of the mouse DNA ligase IV gene, in fusion with the Venus variant of GFP. The DNA ligase IV coding region was amplified by PCR with primers lig4-1 (SEQ ID NO: 17) and lig4-2 (SEQ ID NO: 18) from cDNA clone FANTOM-4932416F16 (obtained from Imagenes GmbH, Berlin) and ligated into the MluI site of plasmid pCAG-venus-Mlu (R. Kuhn, unpublished).

Alternatively we used a synthetic peptide (Lig4-759) comprising residues 759-814 of the mouse DNA ligase IV protein (FIG. 7) (SEQ ID NO: 19) that mimics the binding site of DNA ligase IV to XRCC4 (21), able to interfere with the formation of native DNA ligase IV/XRCC4 complexes. To directly inhibit the enzymatic activity of DNA ligase IV we used the DNA ligase inhibitor L189 (24) (6-Amino-2,3-dihydro-5-[(phenylmethylene)amino]-2-4(1H)-py rimidine one; CAS No 64232-83-3; Tocris Bioscience, Ellisville, USA).

In addition, we constructed the plasmids pCAG-E4ORF6-bpA (FIG. 7 D) (SEQ ID NO: 20), containing the complete coding sequence (1-294) of the Adenovirus-5 E4ORF6 protein, and pCAG-E1b55K-bpA (FIG. 7E) (SEQ ID NO: 21), containing the complete coding sequence (1-496) of the Adenovirus-5 E1b (55K) protein. The adenovirus E4ORF6 (34k) and E1b-55k proteins target host DNA ligase IV for proteasomal degradation and thereby inhibit NHEJ DNA repair (25-28). The E4ORF6 coding region was amplified by PCR with primers E4-1 (SEQ ID NO: 22) and E4-2 (SEQ ID NO: 23) from plasmid pHelper (Stratagene). The coding region of E1b55K was amplified by PCR with primers E1b-1 (SEQ ID NO: 24) and E1b-2 (SEQ ID NO: 25) from genomic DNA of AAV-293 cells (Stratagene). The PCR products were ligated into the PacI and MluI sites of plasmid pCAG-Cre-pA (R. Kahn, unpublished).

To interfere with the activity of the Ku70/Ku80 complex in the pronucleus of fertilised oocytes, we constructed the plasmids pCAG-Ku70-bpA (FIG. 7 B) (SEQ ID NO: 26) and pCAG-Ku80-bpA (FIG. 7 C) (SEQ ID NO: 27) that contain the C-terminal part (residues 62-609) of the coding region of the mouse Ku70 gene, or the C-terminal part (residues 427-732) of the coding region of the mouse Ku80 gene. The Ku70 coding region was amplified by PCR with primers Ku70-1 (SEQ ID NO: 28) and Ku70-2 (SEQ ID NO: 29) from cDNA clone IRAVp968D0945D (obtained from Imagenes GmbH, Berlin) and the Ku80 coding region was amplified by PCR with primers Ku80-1 (SEQ ID NO: 30) and Ku80-2 (SEQ ID NO: 31) from cDNA clone IRAVp968E03106D (obtained from Imagenes GmbH, Berlin). The PCR products were ligated into the PacI and MluI sites of plasmid pCAG-Cre-bpA (R. Kuhn, unpublished).

The T7 promoter region located upstream of the coding regions of the pCAG plasmids enabled the production of mRNA as described (13) upon linearization at the end of the coding region with MluI. Purified mRNA (3 ng/μl) coding for DNA ligase IV (652-911) or Ku70 (62-609) or Ku80 (427-732) or full length E4ORF6 and E1b55K were coinjected into zygotes together with ZFN-Rab38L and -R mRNA (3 ng/μl, each) and circular Rab38-cht targeting vector (15 ng/μl).

Alternatively, fertilised oocytes were microinjected with ZFN-Rab38L and -R mRNA (3 ng/μl, each) and circular Rab38-cht targeting vector (15 ng/μl), together with varying amounts of the lig4-759 inhibitory peptide or the L189 inhibitor.

The microinjected zygotes were transferred into pseudopregnant females to allow their further development into live mice. From the resulting mice genomic DNA was extracted from tail tips to analyse for the presence of the desired homologous recombination event at the Rab38 locus by PCR, as described above.

This analysis revealed that individuals harboured one allele of the preplanned Rab38 chocolate mutation were obtained at significantly higher rates as compared to microinjections performed in experiment B (see above) without inhibition of NHEJ DNA repair. Therefore, it is possible to improve a preplanned modification into the coding region of the Rab38 gene by zinc-finger nuclease assisted homologous recombination in fertilised oocytes, provided that the key enzymes DNA ligase IV of Ku70/80 are inhibited in their action.

### EXAMPLE 2

In this example, the frequency of homologous recombination repair following a TAL-nuclease induced double-strand break within a genomic integrated reporter construct in a mammalian cell line was detected. The efficiency of repair is two-fold increased by the coexpression of a truncated Ku80 protein designed to inhibit the function of NHEJ repair.

A) Construction of TAL-Nuclease and Recombination Reporter Vectors

For the expression of Tal nucleases in mammalian cells the generic expression vector pCAG-Tal-IX-Fok (SEQ ID NO: 32) (FIG. 9) was designed, that contains a CAG hybrid promoter region and a transcriptional unit comprising a sequence coding for the N-terminal amino acids 1-176 of Tal nucleases, located upstream of a pair of BsmBI restriction sites. This N-terminal regions includes an ATG start codon, a nuclear localisation sequence, a FLAG Tag sequence, a glycine rich linker sequence, a segment coding for 110 amino acids of the Tal protein AvrBs3 and the invariable N-terminal Tal repeat of the Hax3 Tal effector. Downstream of the central BsmBI sites, the transcriptional unit contains 78 codons including an invariable C-terminal Tal repeat (34 amino acids) and 44 residues derived from the Tal protein AvrBs3, followed by the coding sequence of the FokI nuclease domain and a polyadenylation signal sequence (bpA). DNA segments coding for arrays of Tal repeats, designed to bind a Tal nuclease target sequence can be inserted into the BsmBI sites of pCAG-Tal-IX-Fok in frame with the up- and downstream coding regions to enable the expression of predesigned Tal-Fok nuclease proteins. To generate Tal nuclease vectors against a target region within exon 1 of the mouse Rab38 gene we inserted synthetic DNA segments with the coding regions of two different arrays of Tal repeats (FIG. 9 A-B) into the BsmBI sites of pCAG-Tal-IX-Fok. The expression vectors pCAG-RabChtTal-1 (SEQ ID NO: 33) and pCAG-RabChtTal-2 (SEQ ID NO: 34) enable to express the Tal nuclease proteins RabChtTal1 (SEQ ID NO: 35) and RabChtTal-2 (SEQ ID NO: 36). Together the two nuclease proteins are able to bind to a target region that is derived from exon 1 of the mouse Rab38 gene (FIG. 10). The target sequences were selected such that the binding regions of the Tal nuclease proteins are preceeded by a T nucleotide. Following the sequence downstream of the initial T in the 5′>3′ direction, specific Tal DNA-binding domains were combined together into arrays of 14 Tal elements (FIG. 9). Each Tal element motif consists of 34 amino acids, the position 12 and 13 of which determines the specificity towards recognition of A, G, C or T within the target sequence To derive Tal element DNA-binding domains the Tal effector motif (repeat) #11 of the Xanthomonas Hax3 protein (GenBank accession No. AY993938.1 (LTPEQWAIASNIGGKQALETVQRLLPVLCQAHG (SEQ ID NO: 38)) with amino acids N12 and I13, or S13 to recognize A, the Tal effector motif (repeat) #5 (LTPQQWAIASHDGGKQALETVQRLLPVLCQAHG (SEQ ID NO: 39)) derived from the Hax3 protein with amino acids H12 and D13 to recognize C, and the Tal effector motif (repeat) #4 (LTPQQWAIASNGGGKQALETVQRLLPVLCQAHG (SEQ ID NO: 40)) from the Xanthomonas Hax4 protein (Genbank accession No.: AY993939.1) with amino acids N12 and G13 to recognize T. To recognize a target G nucleotide the Tal effector motif (repeat) #4 from the Hax4 protein with replacement of the amino acids 12 into N and 13 into N (LTPQQWAIASNNGGKQALETVQRLLPVLCQAHG (SEQ ID NO: 41)) was used.

To determine the activity and specificity of the Tal nucleases in mammalian cells a Tal nuclease reporter plasmid was constructed that contains the RabChtTal-1 and RabChtTal-2 target sequences, separated by a 15 bp spacer region (FIG. 10). This configuration enables to measure the activity of a Tal nuclease complex that interacts as a heterodimer of two protein molecules that are bound to the pair of target sequences within the reporter plasmid. Upon DNA binding and interaction of the FokI nuclease domains the reporter plasmid DNA is cleaved within the 15 bp spacer region and exhibits a double-strand break. The Tal nuclease reporter plasmid contains a CMV promoter region, a 400 bp sequence coding for the N-terminal segment of 6-galactosidase and a stop codon. This unit is followed by the Tal nuclease target region. Within the reporter plasmid pCMV-Rab-Reporter(hygro) (SEQ ID NO: 37), the Tal nuclease target region is followed by the complete coding region for β-galactosidase (fused to a neomycin resistance gene) and a polyadenylation signal (pA). In addition the reporter plasmid contains a hygromycin resistance gene. Upon expression of the Tal nuclease protein the reporter plasmid is opened by a nuclease-induced double-strand break within the Tal nuclease target sequence (FIG. 10 A). The DNA regions adjacent to the double-strand break are identical over 400 bp and can be aligned and recombined by homologous recombination DNA repair (FIG. 10 B). Homologous recombination of an opened reporter plasmid will subsequently result into a functional β-galactosidase coding region transcribed from the CMV promoter that leads to the production of β-galactosidase protein (FIG. 10 C). If the double-strand break is closed by NHEJ repair the reporter gene is not reconstituted. Therefore, in a typical cell line the repair pathways of homologous recombination and NHEJ compete for processing of the reporter construct. We assume that the inhibition of NHEJ pathway proteins will lead to an increased number of cells that repair the reporter by homologous recombination. This increase can be quantified by the detection of cells expressing the reporter gene.

To generate a cell line harboring the reporter construct in its genome, linearized plasmid DNA was electroporated into human HEK 293 cells (ATCC #CRL-1573) (Graham F L, Smiley J, Russell W C, Nairn R., J. Gen. Virol. 36, 59-74, 1977) and hygromycin resistant clones were selected and isolated. One of the resistant clones, that showed no background activity of the reporter gene, 293Rab-Rep#4, was selected for further work.

B) Inhibition of Ku80 Increases Nuclease-Induced Recombination in Human Reporter Cells

To evaluate the effect of the inhibition of the NHEJ protein Ku80 one million 293Rab-Rep#4 reporter cells were transfected with 5 μg plasmid DNA of each of the Tal nuclease expression vectors (FIG. 9) together with 5 μg of the unrelated cloning vector pBluescript, or with 5 μg of the plasmid pCAG-Ku80(427-732)-bpA for coexpression of the truncated Ku80 protein. Upon transfection the cells were seeded in duplicate wells of a 6-well tissue culture plate and cultured for two days before analysis was started. For analysis the transfected cells of each well were fixed for 10 minutes with 4% formaldehyde and incubated for 4 hours with X-Gal staining solution (5 mM K3(FeIII(CN)6), 5 mM K4(FeII(CN)6), 2 mM MgCl2, 1 mg/ml X-Gal (5-bromo-chloro-3-indoyl-β-D-galactopyranosid). Recombined cells that express the reporter gene are visualized by an intracellular blue staining and were quantified on photographic images using the ImageJ software's cell counter function (http://imagej.nih.gov/ij). As shown in FIG. 11 A the transfection of pCAG-RabChtTal-1 and pCAG-RabChtTal-2 resulted into a fraction of homologous recombined cells that express the reporter gene. As quantified from two images, 4.89% of the reporter cells (139 positive cells of 2838 cells) showed successful recombination as indicated by expression of the reporter gene. As shown in FIG. 11 B cotransfection of the RabChtTal nuclease plasmids with pCAG-Ku80(427-732)-bpA resulted in a substantial increase of cells harboring successful recombination events. Cells were counted from two representative images indicating a recombination rate of 9.50% (270 positive cells of 2842 cells in total). In conclusion, this result indicates that the suppression of Ku80 function leads a two-fold increase of the rate of cells that repair a nuclease-induced double-strand break by homologous recombination. Therefore the inhibition of Ku80 facilitates the generation and isolation of mammalian cells harboring homologous recombination events.

### REFERENCES

1. Capecchi M R (2005) Gene targeting in mice: functional analysis of the mammalian genome for the twenty-first century. (Translated from eng) Nat Rev Genet. 6(6):507-512 (in eng).
2. Palmiter R D & Brinster R L (1985) Transgenic mice. (Translated from eng) Cell 41(2):343-345 (in eng).
3. Brinster R L, Braun R E, Lo D, Avarbock M R, Oram F, & Palmiter R D (1989) Targeted correction of a major histocompatibility class II E alpha gene by DNA microinjected into mouse eggs. (Translated from eng) Proc Natl Acad Sci USA 86(18):7087-7091 (in eng).
4. Porteus M H & Carroll D (2005) Gene targeting using zinc finger nucleases. (Translated from eng) Nat Biotechnol 23(8):967-973 (in eng).
5. Doyon Y, McCammon J M, Miller J C, Faraji F, Ngo C, Katibah G E, Amora R, Hocking T D, Zhang L, Rebar E J, Gregory P D, Urnov F D, & Amacher S L (2008) Heritable targeted gene disruption in zebrafish using designed zinc-finger nucleases. (Translated from eng) Nat Biotechnol 26(6):702-708 (in eng).
6. Geurts A M, Cost G J, Freyvert Y, Zeitler B, Miller J C, Choi V M, Jenkins S S, Wood A, Cui X, Meng X, Vincent A, Lam S, Michalkiewicz M, Schilling R, Foeckler J, Kalloway S, Weiler H, Menoret S, Anegon I, Davis G D, Zhang L, Rebar E J, Gregory P D, Urnov F D, Jacob H J, & Buelow R (2009) Knockout rats via embryo microinjection of zinc-finger nucleases. (Translated from eng) Science 325(5939):433 (in eng).
7. Rouet P, Smih F, & Jasin M (1994) Expression of a site-specific endonuclease stimulates homologous recombination in mammalian cells. (Translated from eng) Proc Natl Acad Sci USA 91(13):6064-6068 (in eng).
8. Hockemeyer D, Soldner F, Beard C, Gao Q, Mitalipova M, DeKelver R C, Katibah G E, Amora R, Boydston E A, Zeitler B, Meng X, Miller J C, Zhang L, Rebar E J, Gregory P D, Urnov F D, & Jaenisch R (2009) Efficient targeting of expressed and silent genes in human ESCs and iPSCs using zinc-finger nucleases. (Translated from eng) Nat Biotechnol 27(9):851-857 (in eng).
9. Miller J C, Tan S, Qiao G, Barlow K A, Wang J, Xia D F, Meng X, Paschon D E, Leung E, Hinkley S J, Dulay G P, Hua K L, Ankoudinova I, Cost G J, Urnov F D, Zhang H S, Holmes M C, Zhang L, Gregory P D, & Rebar E J (A TALE nuclease architecture for efficient genome editing. (Translated from Eng) Nat Biotechnol (in Eng).
10. Meyer M, de Angelis M H, Wurst W, & Kuhn R (2010) Gene targeting by homologous recombination in mouse zygotes mediated by zinc-finger nucleases. (Translated from eng) Proc Natl Acad Sci USA 107(34):15022-15026 (in eng).
11. Cui X, Ji D, Fisher D A, Wu Y, Briner D M, & Weinstein E J (Targeted integration in rat and mouse embryos with zinc-finger nucleases. (Translated from eng) Nat Biotechnol 29(1):64-67 (in eng).
12. Carbery I D, Ji D, Harrington A, Brown V, Weinstein E J, Liaw L, & Cui X (Targeted genome modification in mice using zinc-finger nucleases. (Translated from eng) Genetics 186(2):451-459 (in eng).
13. Meyer M, de Angelis M H, Wurst W, & Kuhn R (Gene targeting by homologous recombination in mouse zygotes mediated by zinc-finger nucleases. (Translated from eng) Proc Natl Acad Sci USA 107(34):15022-15026 (in eng).
14. Lieber M R (The mechanism of double-strand DNA break repair by the nonhomologous DNA end-joining pathway. (Translated from eng) Annu Rev Biochem 79:181-211 (in eng).
15. Bozas A, Beumer K J, Trautman J K, & Carroll D (2009) Genetic analysis of zinc-finger nuclease-induced gene targeting in Drosophila. (Translated from eng) Genetics 182(3):641-651 (in eng).
16. Frank K M, Sharpless N E, Gao Y, Sekiguchi J M, Ferguson D O, Zhu C, Manis J P, Homer J, DePinho R A, & Alt F W (2000) DNA ligase IV deficiency in mice leads to defective neurogenesis and embryonic lethality via the p53 pathway. (Translated from eng) Mol Cell 5(6):993-1002 (in eng).
17. Forrester N A, Sedgwick G G, Thomas A, Blackford A N, Speiseder T, Dobner T, Byrd P J, Stewart G S, Turnell A S, & Grand R J (2011) Serotype-specific inactivation of the cellular DNA damage response during adenovirus infection. (Translated from eng) J Virol 85(5):2201-2211 (in eng).
18. Cheng C Y, Gilson T, Dallaire F, Ketner G, Branton P E, & Blanchette P (2011) The E4orf6/E1B55K E3 ubiquitin ligase complexes of human adenoviruses exhibit heterogeneity in composition and substrate specificity. (Translated from eng) J Virol 85(2):765-775 (in eng).
19. Loftus S K, Larson D M, Baxter L L, Antonellis A, Chen Y, Wu X, Jiang Y, Bittner M, Hammer J A, 3rd, & Pavan W J (2002) Mutation of melanosome protein RAB38 in chocolate mice. (Translated from eng) Proc Natl Acad Sci USA 99(7):4471-4476 (in eng).
20. Ellenberger T & Tomkinson A E (2008) Eukaryotic DNA ligases: structural and functional insights. (Translated from eng) Annu Rev Biochem 77:313-338 (in eng).
21. Wu P Y, Frit P, Meesala S, Dauvillier S, Modesti M, Andres S N, Huang Y, Sekiguchi J, Calsou P, Salles B, & Junop M S (2009) Structural and functional interaction between the human DNA repair proteins DNA ligase IV and XRCC4. (Translated from eng) Mol Cell Biol 29(11):3163-3172 (in eng).
22. He F, Li L, Kim D, Wen B, Deng X, Gutin P H, Ling C C, & Li G C (2007) Adenovirus-mediated expression of a dominant negative Ku70 fragment radiosensitizes human tumor cells under aerobic and hypoxic conditions. (Translated from eng) Cancer Res 67(2):634-642 (in eng).
23. Osipovich O, Duhe R J, Hasty P, Durum S K, & Muegge K (1999) Defining functional domains of Ku80: DNA end binding and survival after radiation. (Translated from eng) Biochem Biophys Res Commun 261(3):802-807 (in eng).
24. Chen X, Zhong S, Zhu X, Dziegielewska B, Ellenberger T, Wilson G M, MacKerell AD, Jr., & Tomkinson A E (2008) Rational design of human DNA ligase inhibitors that target cellular DNA replication and repair. (Translated from eng) Cancer Res 68(9):3169-3177 (in eng).
25. Baker A, Rohleder K J, Hanakahi L A, & Ketner G (2007) Adenovirus E4 34k and E1b 55k oncoproteins target host DNA ligase IV for proteasomal degradation. (Translated from eng) J Virol 81(13):7034-7040 (in eng).
26. Jayaram S, Gilson T, Ehrlich E S, Yu X F, Ketner G, & Hanakahi L (2008) E1B 55k-independent dissociation of the DNA ligase IV/XRCC4 complex by E4 34k during adenovirus infection. (Translated from eng) Virology 382(2):163-170 (in eng).
27. Mohammadi E S, Ketner E A, Johns D C, & Ketner G (2004) Expression of the adenovirus E4 34k oncoprotein inhibits repair of double strand breaks in the cellular genome of a 293-based inducible cell line. (Translated from eng) Nucleic Acids Res 32(8):2652-2659 (in eng).
28. Hart L S, Yannone S M, Naczki C, Orlando J S, Waters S B, Akman S A, Chen D J, Ornelles D, & Koumenis C (2005) The adenovirus E4orf6 protein inhibits DNA double strand break repair and radiosensitizes human tumor cells in an E1B-55K-independent manner. (Translated from eng) J Biol Chem 280(2):1474-1481 (in eng).

1. A method for modifying a target sequence in the genome of a mammalian cell, the method comprising the step of introducing into a mammalian cell:
(a) one or more compounds that introduce double-strand breaks in said target sequence;
(b) one or more DNA molecules comprising a donor DNA sequence to be incorporated by homologous recombination into the genomic DNA of said mammalian cell within said target sequence, wherein said donor DNA sequence is flanked upstream by a first flanking element and downstream by a second flanking element, wherein said first and second flanking element are different and wherein each of said first and second flanking element are homologous to a continuous DNA sequence on either side of the double-strand break introduced by said one or more compounds of (a) within said target sequence in the genome of said mammalian cell; and
(c) one or more compounds that decrease the activity of the non-homologous end joining (NHEJ) DNA repair complex in said mammalian cell.

2. The method of claim 1, wherein said one or more compounds in (a) are selected from the group consisting of TAL nucleases; zinc-finger nucleases; engineered meganucleases; nucleic acid molecules encoding said TAL nucleases in expressible form; zinc-finger nucleases in expressible form; and engineered meganucleases in expressible form.

3. The method of claim 2, wherein the zinc-finger nucleases or TAL nucleases are fusion (poly)peptides of target sequence specific zinc-finger or TAL DNA binding domains and:
a (poly)peptide comprising or consisting of the cleavage domain of the FokI endonuclease; or
a (poly)peptide that is encoded by a nucleic acid molecule encoding:
(I) a (poly)peptide having the activity of an endonuclease, which is (i) a nucleic acid molecule encoding a (poly)peptide comprising or consisting of the amino acid sequence of SEQ ID NO: 5; (ii) a nucleic acid molecule comprising or consisting of the nucleotide sequence of SEQ ID NO: 6; (iii) a nucleic acid molecule encoding an endonuclease, the amino acid sequence of which is at least 70% identical to the amino acid sequence of SEQ ID NO: 5; (iv) a nucleic acid molecule comprising or consisting of a nucleotide sequence which is at least 50% identical to the nucleotide sequence of SEQ ID NO: 6; (v) a nucleic acid molecule which is degenerate with respect to the nucleic acid molecule of (iv); or (vi) a nucleic acid molecule corresponding to the nucleic acid molecule of any one of (i) to (v) wherein T is replaced by U; or
(II) a fragment of the (poly)peptide of (I) having the activity of an endonuclease.

4. The method of claim 1, wherein the activity of said NHEJ DNA repair complex in (c) is decreased by decreasing the activity of NHEJ DNA ligase IV (LIG4).

5. The method of claim 4, wherein the one or more compounds that decrease the activity of the non-homologous end joining (NHEJ) DNA repair complex are selected from the group consisting of small molecules, RNAi-molecules, antisense nucleic acid molecules, ribozymes, compounds inhibiting the formation of a functional LIG4 complex and compounds enhancing proteolytic degradation of a functional LIG4 complex.

6. The method of claim 5, wherein a small molecule comprises 6-Amino-2,3-dihydro-5-[(phenylmethylene)]amino]-2-4(1H)-pyrimidineone).

7. The method of claim 5, wherein the formation of a functional LIG4 complex can be inhibited by compounds that inhibit the binding of LIG4 to XRCC4 or inhibit the binding of Ku70 to Ku80.

8. The method of claim 7, wherein said compounds inhibiting the binding of LIG4 to XRCC4 or inhibiting the binding of Ku70 to Ku80 comprise (poly)peptides or nucleic acids encoding said (poly)peptides.

9. The method of claim 8, wherein said (poly)peptides inhibiting the binding of
L1G4 to XRCC4 are the binding domains of LIG4 or XRCC4 mediating the binding of LIG4 to XRCC4; and the polypeptides inhibiting the binding of
Ku70 to Ku80 are the binding domains of Ku70 or Ku80 mediating the binding of Ku70 to Ku80.

10. The method of claim 5, wherein said compounds enhancing proteolytic degradation of LIG4 comprise adenoviral (poly)peptides E1b55K and E4ORF6.

11. The method of claim 10, wherein said adenoviral (poly)peptides have been derived from a human adenovirus of serotype Ad9 or Ad16.

12. The method of claim 1, wherein said mammalian cell is selected from the group consisting of an ungulate cell, a rodent cell, a rabbit cell, a primate cell or a human cell.

13. The method of claim 1, wherein the mammalian cell is a mouse or a rat cell.

14. The method of claim 1 wherein the mammalian cell is an embryonic stem cell or an oocyte.

15. A method of producing a non-human mammal carrying a modified target sequence in its genome, the method comprising transferring a cell produced by the method of claim 1 into a pseudo pregnant female host.
